(19) United States
(12) Patent Application Publication
(10) Pub. No.: US 2024/0182852 A1
(43) Pub. Date: Jun. 6, 2024

(54) CELL-CULTURE BIOREACTORS

(71) Applicant: Orgenesis Inc., Germantown, MD (US)

(72) Inventors: Michael FAINSHTEIN, Kazan (RU); Nurislam GUBAEV, KAZAN (RU); Vered CAPLAN, Coppet (CH)

(73) Assignee: Orgenesis Inc., Germantown, MD (US)

(21) Appl. No.: 18/556,083

(22) PCT Filed: Apr. 19, 2022
(86) PCT No.: PCT/US2022/025407

§ 371 Date: Oct. 18, 2023

Related U.S. Application Data
(60) Provisional application No. 63/177,317, filed on Apr. 20, 2021.

(51) Int. Cl. C12N 5/0783; C12M 1/00; C12M 1/26

(52) U.S. Cl. CPC C12N 5/0636; C12M 23/34; C12M 23/40; C12M 29/06; C12M 29/14; C12M 33/04

(57) ABSTRACT

A bioreactor is provided, comprising a housing extending along a vertical axis and defining an internal cavity therewithin, top and bottom covers at opposite ends of the housing, and a plurality of ports, each configured to facilitate fluid communication between the internal cavity and the exterior of the bioreactor. The internal cavity has a frustoconical shape, the frustoconical shape being formed such that horizontal cross-sections are coaxial about a line which is angled with respect to the vertical axis.

### CROSS-REFERENCE TO RELATED APPLICATIONS

This application claims benefit of U.S. Provisional Patent Application No. 63/177,317, filed Apr. 20, 2021, which is incorporated by reference herein in its entirety.

### FIELD OF THE INVENTION

The presently disclosed subject matter is directed to bioreactors for carrying out bioprocesses therein, and to systems, methods, and auxiliary apparatuses associated with bioreactors for carrying out bioprocesses.

### BACKGROUND

Cell culture generally involves the removal of cells from an animal or plant and culturing the cells in a favorable artificial environment. Culture conditions vary widely for each cell type, but the artificial environment in which the cells are cultured typically involves the use of a suitable vessel containing: a substrate or medium that supplies the essential nutrients (such as amino acids, carbohydrates, vitamins, minerals); growth factors; hormones; gases (such as O₂, CO₂); and a regulated physio-chemical environment (including regulation of pH, osmotic pressure, temperature, etc.). Some cells must be cultured while attached to a solid or semi-solid substrate (adherent or monolayer culture), while others can be grown floating in the culture medium (suspension culture). The expanded cell cultures can then be used in further bioprocessing in the production of therapeutic products.

Perfusion involves upstream processing which retains cells inside the bioreactor while continually removing cell waste products and media depleted of nutrients by cell metabolism. Fresh media is provided to the cells at the same rate as the spent media is removed. By continuously removing spent media and replacing it with new media, nutrient levels are maintained for optimal growing conditions and cell waste product is removed to avoid toxicity. In addition, the product is regularly removed before being exposed to excessive waste that causes protein degradation.

A bioreactor can be employed in a bioprocess to optimize cell culture, with many factors influencing this optimization, including dissolved oxygen (DO) and carbon dioxide (CO₂) levels. Oxygen is crucial for the cellular processes of respiration and cell division and CO₂ is a waste byproduct of these processes. Both DO and CO₂ can impact the culture media pH and the product quality and thus these levels need to be controlled. Within a bioreactor, different mechanical and/or chemical processes may be employed to ensure that DO levels remain within a predetermined range, or at a predetermined value. Care must be taken, however, that these processes do not cause excessive shear stress on the cells being cultured, which may negatively affect cell culture yield.

Moreover, reusable bioreactors can suffer from excess maintenance and operating costs and problematic galvanic and polarographic sensors.

Accordingly, there remains a need in the art for cell culture bioreactors with improved designs that overcome these and other shortcomings of those known in the art.

### SUMMARY

According to an aspect of the presently disclosed subject matter, there is provided a bioreactor comprising:
a housing extending along a vertical axis and defining an internal cavity therewithin, and top and bottom covers at opposite ends of the housing; and
a plurality of ports, each configured to facilitate fluid communication between the internal cavity and the exterior of the bioreactor;
   wherein the internal cavity has a frustoconical shape, the frustoconical shape being formed such that horizontal cross-sections are coaxial about a line which is angled with respect to the vertical axis.

The frustoconical shape of the internal cavity may parallel to the vertical axis along a single line.

The housing may comprise upper and lower openings, each closed by a respective one of the top and bottom covers, and each being perpendicular to the vertical axis.

The housing may comprise a vertically extending window.

At least some of the ports may be configured to facilitate selective fluid communication between the internal cavity and the exterior of the bioreactor.

At least one of the ports may constitute a liquid injection port facilitating injection of a liquid into the internal cavity.

The liquid injection port may comprise a septum membrane providing fluid isolation between the internal cavity and the exterior of the bioreactor, the septum membrane being configured to be facilitate the fluid communication by being pierced by a syringe.

At least one of the ports may constitute a collection port configured to be connected to an external container. The collection port may comprise a tap connected thereto.

At least one of the ports may constitute a gas supply port. The gas supply port may comprise a filter.

At least one of the ports may constitute a needleless access port. The needleless access port may comprise a swabable valve.

At least one of the ports may constitute a liquid supply port. The liquid supply port may comprise a check valve.

The bioreactor may further comprise one or more filter membranes.

One or more of the ports may be formed in the top or bottom cover, the membrane being disposed between the port and the internal cavity.

According to another aspect of the presently disclosed subject matter, there is provided an apparatus comprising:
an upper tank for receiving therewith a liquid medium and a lower tank in fluid communication with the upper tank via a flow passage;
a plurality of ports, each configured to facilitate fluid communication between the interior and the exterior of the apparatus;
a filter membrane disposed such that liquid flowing from the upper tank to the lower tank mast pass therethrough;
a stirring mechanism configured to mix the contents of the upper tank;
an ultrasonic shaker disposed adjacent the filter; and
a controller configured to direct operation of the stirring mechanism and ultrasonic shaker, and to facilitate passage of liquid from the upper tank to the lower tank.

The filter membrane may comprise a reinforced filter.

The filter membrane may be selected from the group including a microfiltration membrane, an ultrafiltration membrane, and a nanofiltration membrane.

The filter may comprise a pore size of 100 kDa NMWCO.

The stirring mechanism may comprises a magnetic stirrer.

The apparatus may further comprise a pump configured to facilitate liquid flow through the system.

According to another aspect of the presently disclosed subject matter, there is provided a method of culturing immune cells comprising:
seeding a population of immune cells in media in the internal cavity of a bioreactor according to any of the above aspects;
activating the immune cells;
transducing the activated immune cells with a viral vector;
expanding the transduced immune cells; and
harvesting the transduced immune cells when the desired concentration of cells is obtained.

The method may further comprise suppling gas through a gas supply port of the bioreactor.

The gas may be selected from nitrogen, carbon dioxide, and oxygen.

The method may further comprise a step of washing the immune cells.

Washing the immune cells may comprise:
tilting the bioreactor toward a horizontal position;
increasing pressure inside the bioreactor with nitrogen and extracting media or buffer through bioreactor waste valve;
decreasing pressure inside the bioreactor and tilting the bioreactor toward a vertical position adding wash buffer; and
optionally, repeating steps (a) to (d).

The method may further comprise adding fresh media and one or more growth factors to the bioreactor.

The method may be characterized in that:
when the bioreactor contains between 300 to 500 ml media, tilting is 83° from the vertical axis;
when the bioreactor contains between 100 ml to 300 ml media, tilting is 73° from the vertical axis;
when the bioreactor contains between 15 ml to 100 ml media, tilting is 61° from the vertical axis; and
when the bioreactor contains up to 15 ml media, tilting is 23° from the vertical axis.

The population of immune cells may comprise a population of T cells.

The cells obtained may comprise CAR T cells configured for adoptive cell therapy.

According to another aspect of the presently disclosed subject matter, there is provided a population of T cells expressing chimeric antigen receptor obtained by the method of the previous aspect.

According to another aspect of the presently disclosed subject matter, there is provided a connection panel configured to facilitate functionally connecting a bioreactor to a control unit, the connection panel being separate from the bioreactor and from the control unit, the connection panel comprising a printed circuit board with a plurality of nodes, each of the nodes being configured for connection to the control unit and connection to an element associated with operation of the bioreactor for functionally connecting it to the control unit.

The connection panel may comprise a printed circuit board with a plurality of nodes, each of the nodes being configured for connection to an element associated with operation of the bioreactor for communication therewith independently of elements connected to other of the nodes.

The nodes may be connected to a single bus, the bus being configured to facilitate connection of the connection panel to the control unit.

The nodes and bus may be provided according to a CAN architecture.

The connection panel may comprise a through-going slot for accommodating therethrough of a grip of the control unit.

The connection panel may comprise a template indicating locations for connections of elements to the connection panel.

The template may be provided on a sheet configured to be removably affixable to the connection panel.

According to another aspect of the presently disclosed subject matter, there is provided a kit for use with a connection panel according to the previous aspect, the kit being configured to facilitate use of the connection panel and control unit with a bioreactor for performing therein a predefined bioprocess, the kit comprising:
the bioreactor;
a plurality of elements associated with operation of the bioreactor to carry out the bioprocess; and
a sheet being removably affixable to the connection panel, the sheet comprising a template thereon, the template indicating, when suitably affixed to the connection panel, points of connections of the elements to the connection panel, wherein connection of the elements to the connection panel as per the template facilitates operation of the elements by the control tower to carry out the predefined bioprocess.

The elements may comprise one or more filters, one or more sensors, and/or one or more tubes.

At least some of the elements may be disposable.

### BRIEF DESCRIPTION OF THE DRA WINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

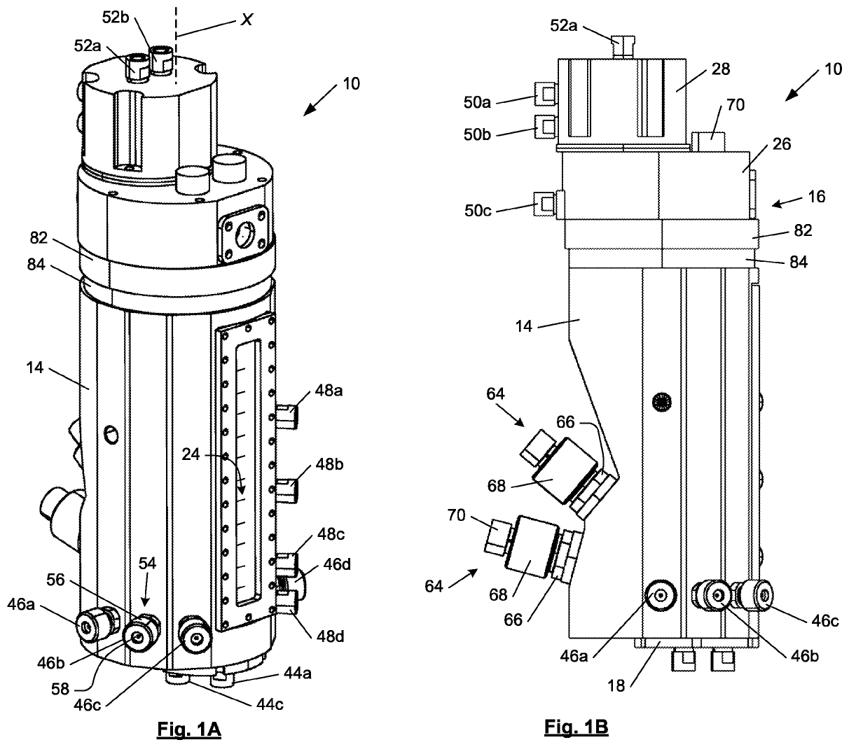

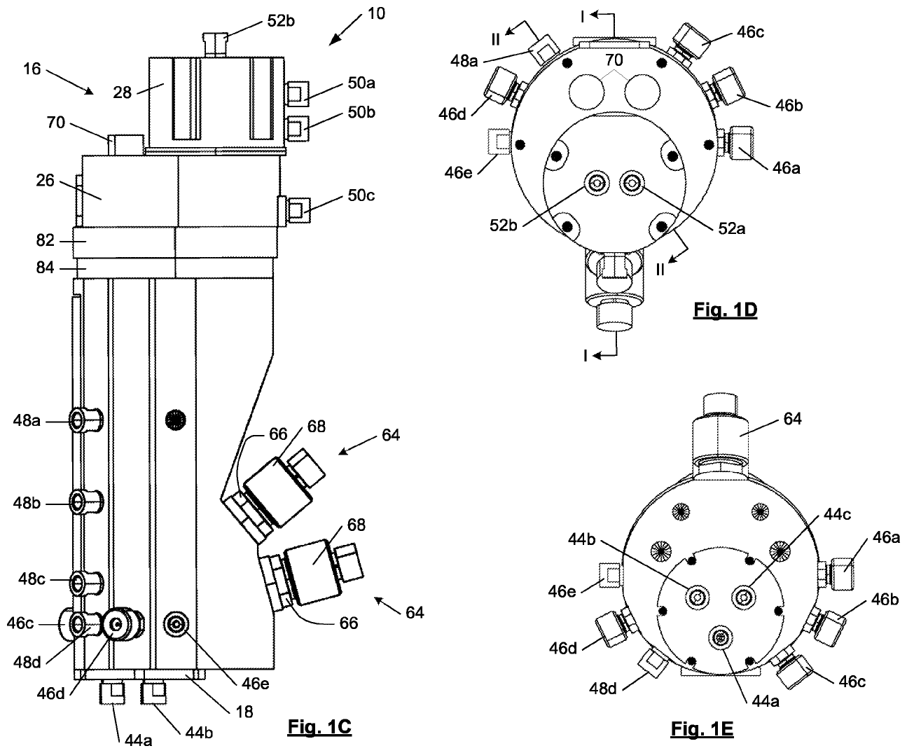

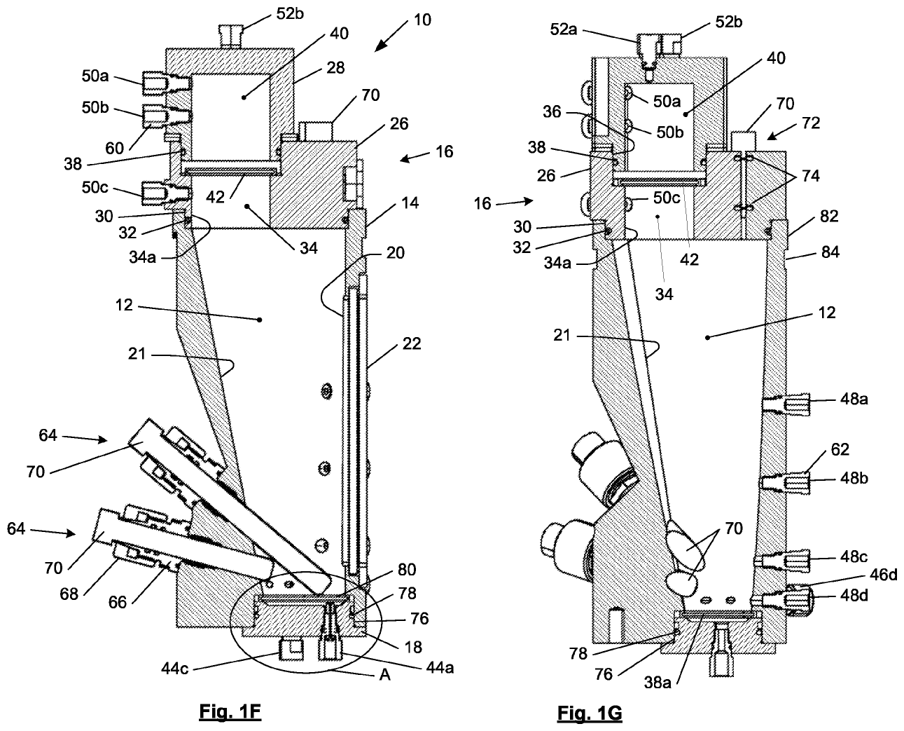

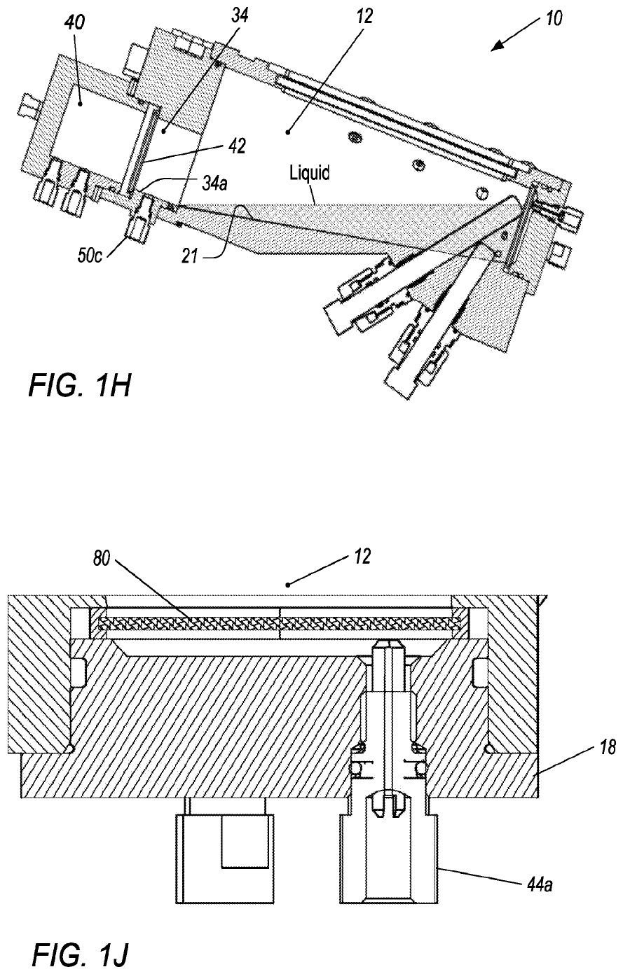

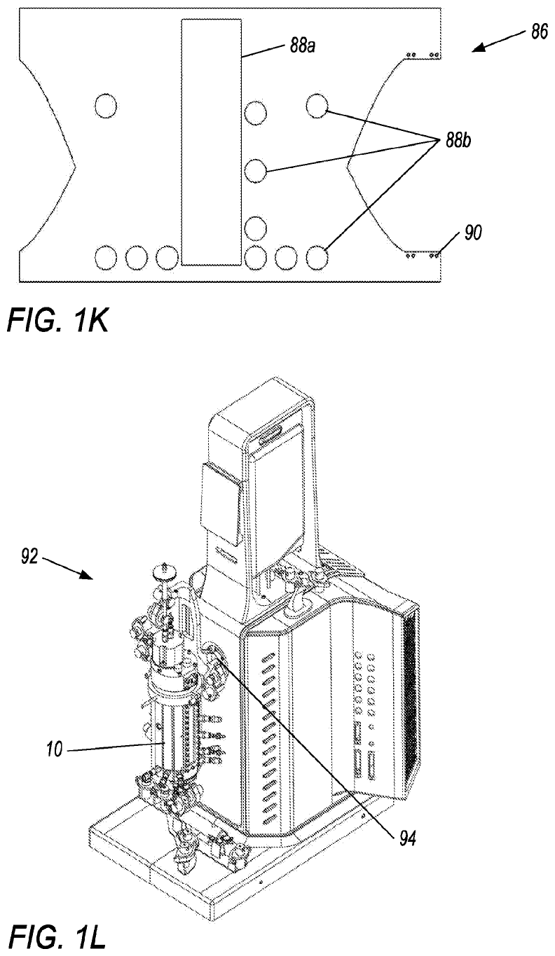

FIG. 1A is a perspective view of an asymmetric bioreactor according to the presently disclosed subject matter;

FIG. 1B is a left side view of the asymmetric bioreactor illustrated in FIG. 1A;

FIG. 1C is a right side view of the asymmetric bioreactor illustrated in FIG. 1A;

FIG. 1D is top view of the asymmetric bioreactor illustrated in FIG. 1A;

FIG. 1E is a bottom view of the asymmetric bioreactor illustrated in FIG. 1A;

FIG. 1F is a cross-sectional view taken along line I-I in FIG. 1D;

FIG. 1G is a cross-sectional view taken along line I-II in FIG. 1D;

FIG. 1H is the cross-sectional view of FIG. 1F, in a tilted position with liquid therewithin;

FIG. 1J is a closeup view of the area indicated at ‘A’ in FIG. 1F;

FIG. 1K illustrates a heating panel for use with the asymmetric bioreactor illustrated in FIGS. 1A through 1H;

FIG. 1L illustrates a portion of a system comprising a control tower and the asymmetric bioreactor illustrated in FIGS. 1A through 1H;

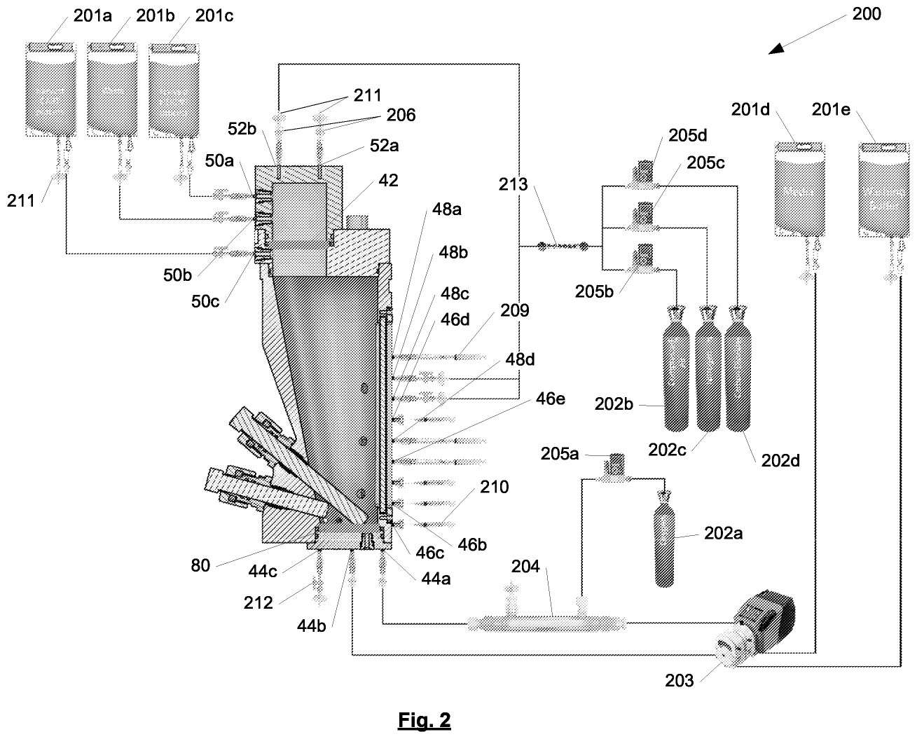

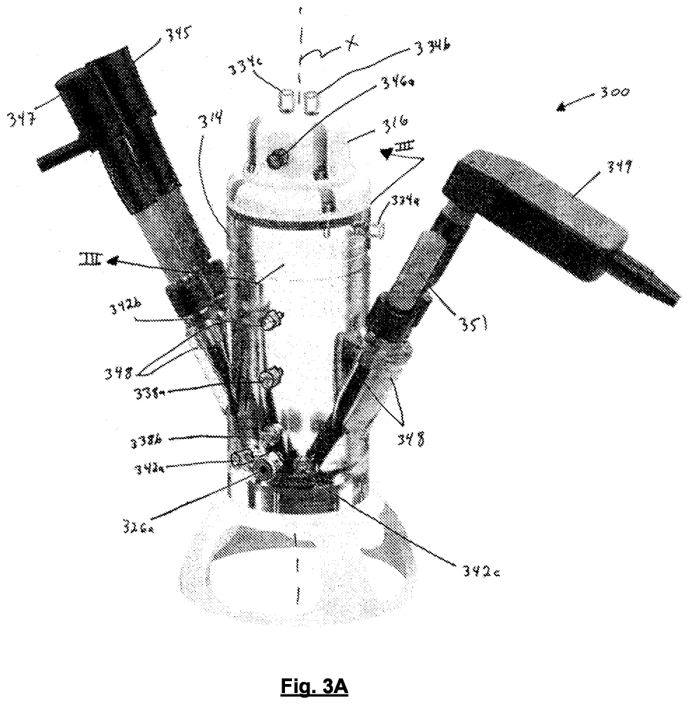

FIG. 2 schematically illustrates a system that includes the asymmetric bioreactor illustrated in FIGS. 1A through 1G and peripheral devices usable with the bioreactor;

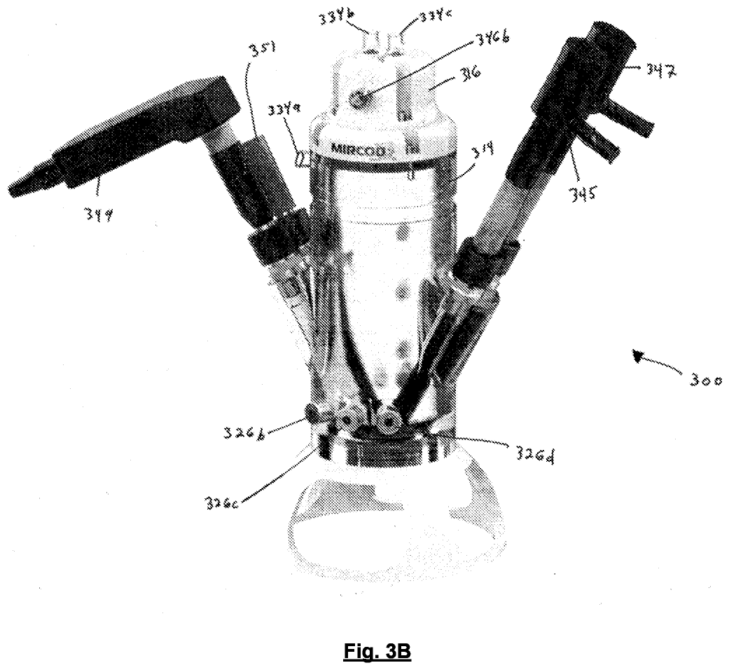

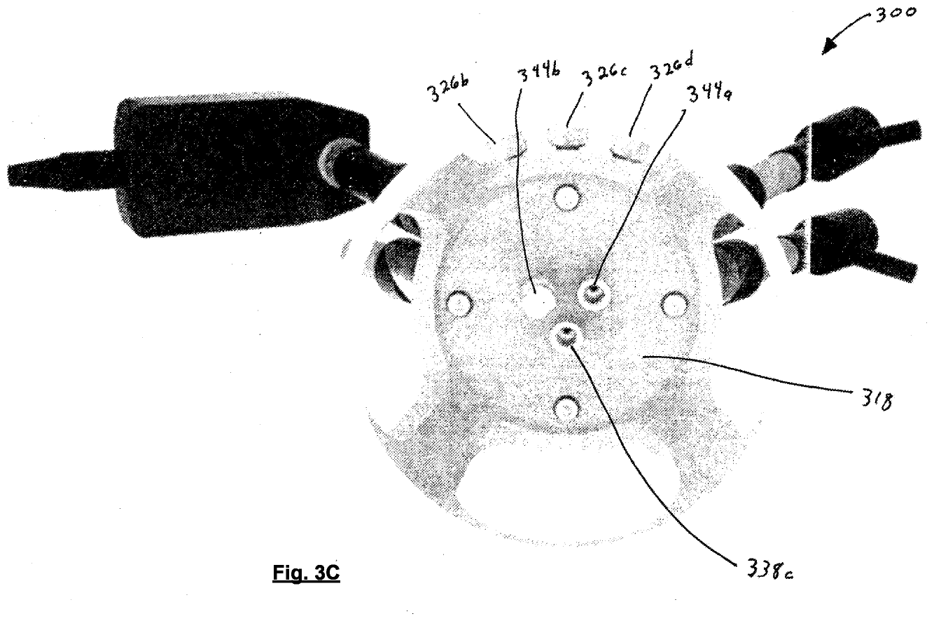

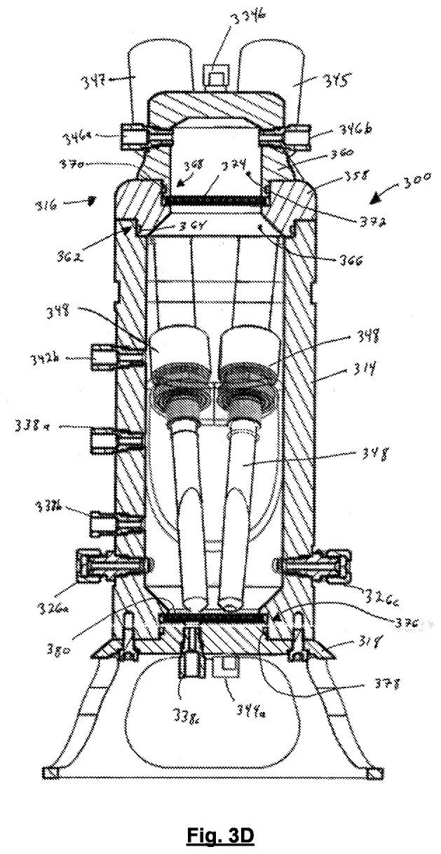

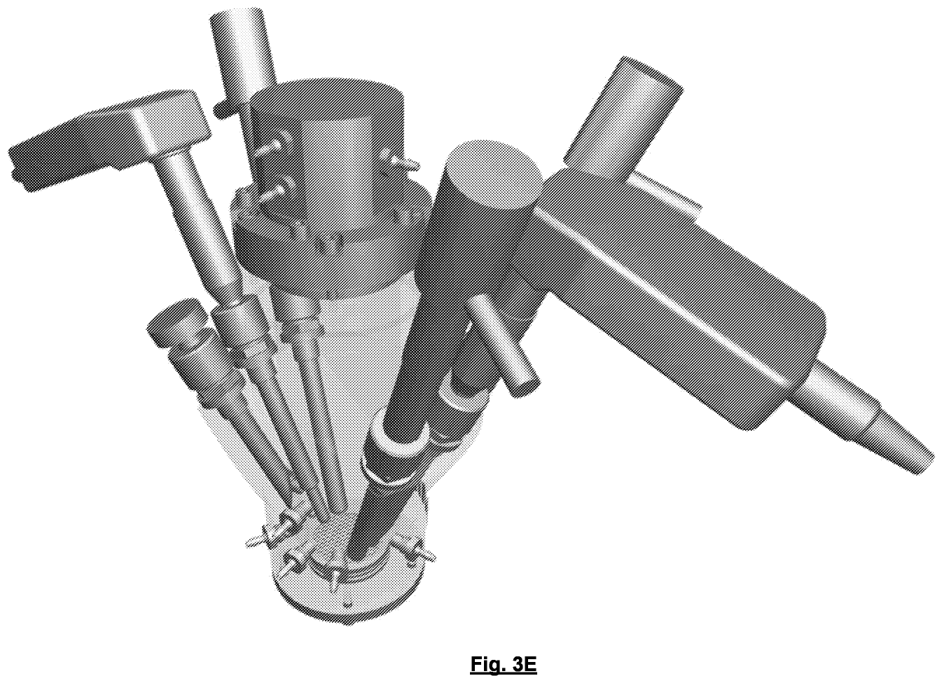

FIG. 3A is a left side perspective view of a cylindrical bioreactor according to the presently disclosed subject matter;

FIG. 3B is a right side view of the cylindrical bioreactor illustrated in FIG. 3A;

FIG. 3C is a bottom view of the cylindrical bioreactor illustrated in FIG. 3A;

FIG. 3D is a cross-sectional view taken along line II-II in FIG. 3A;

FIG. 3E is a perspective view of another example of a cylindrical bioreactor according to the presently disclosed subject matter;

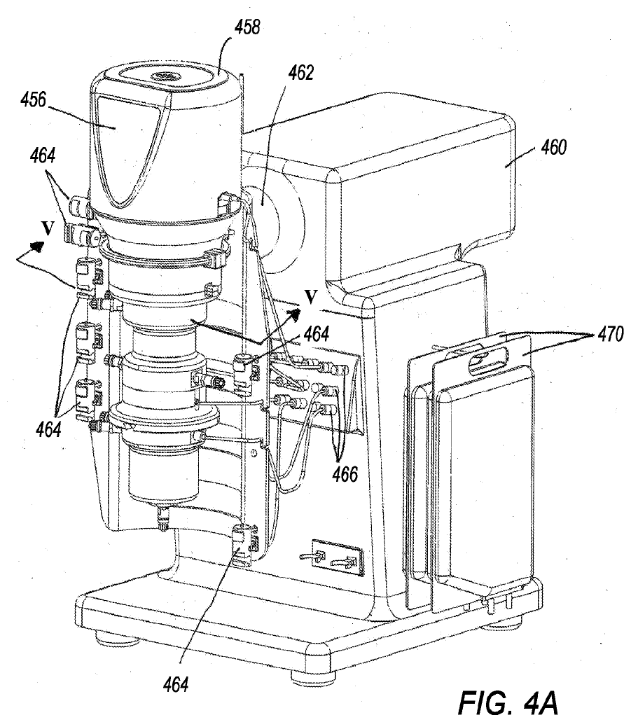

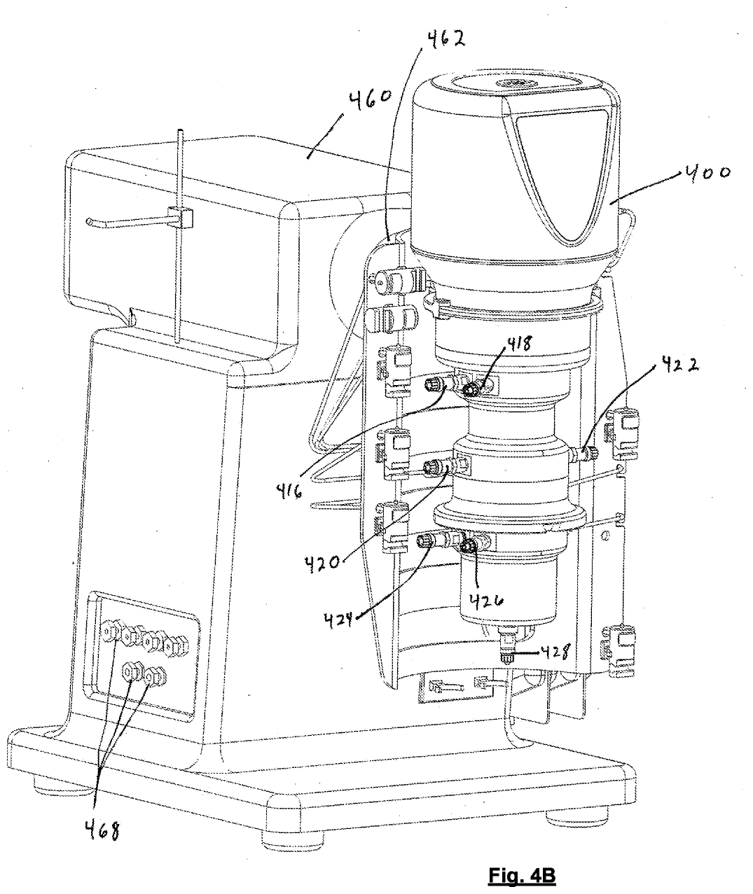

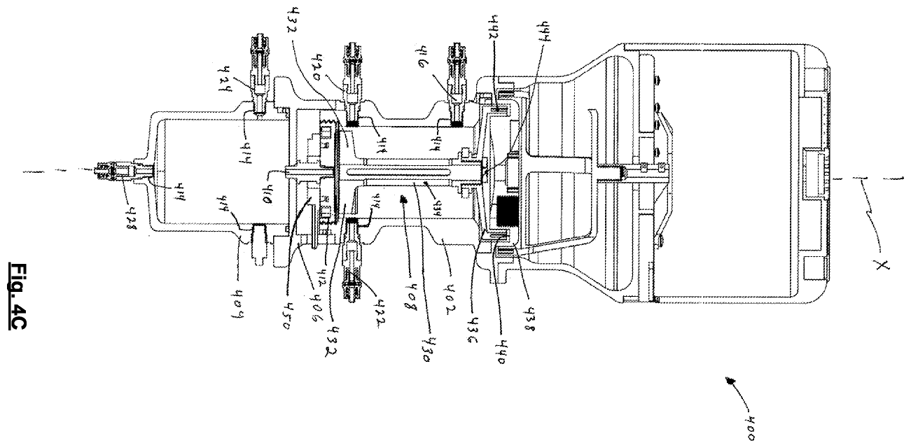

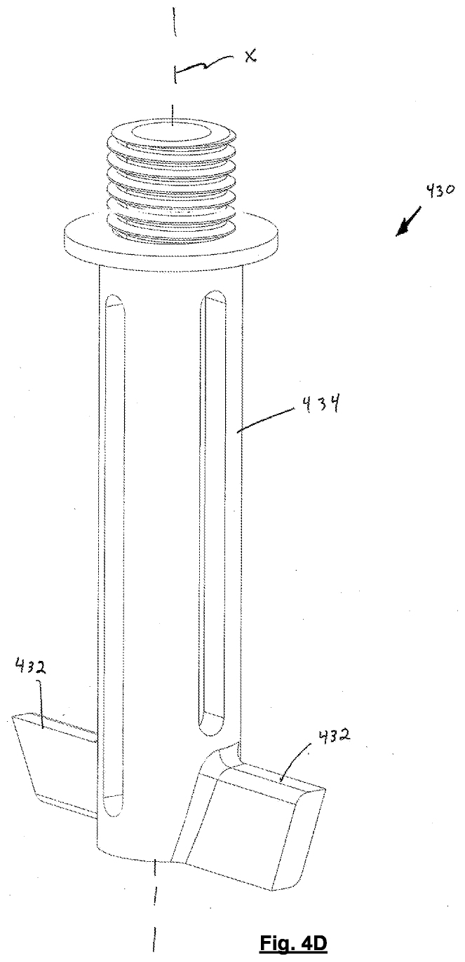

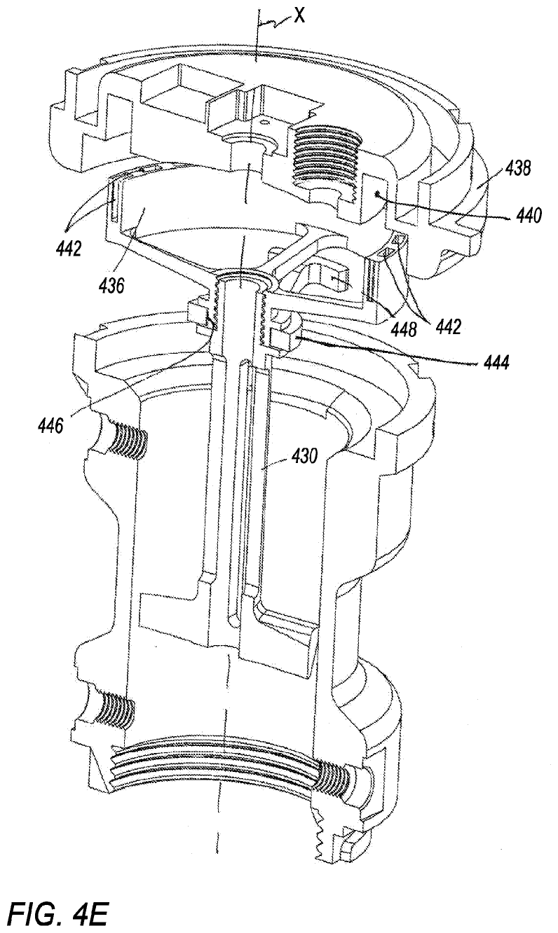

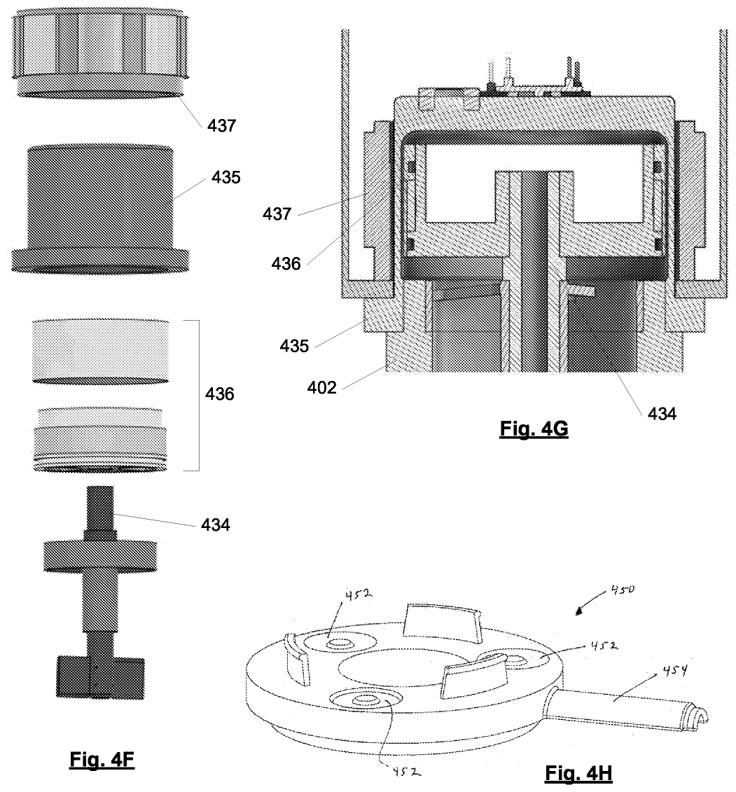

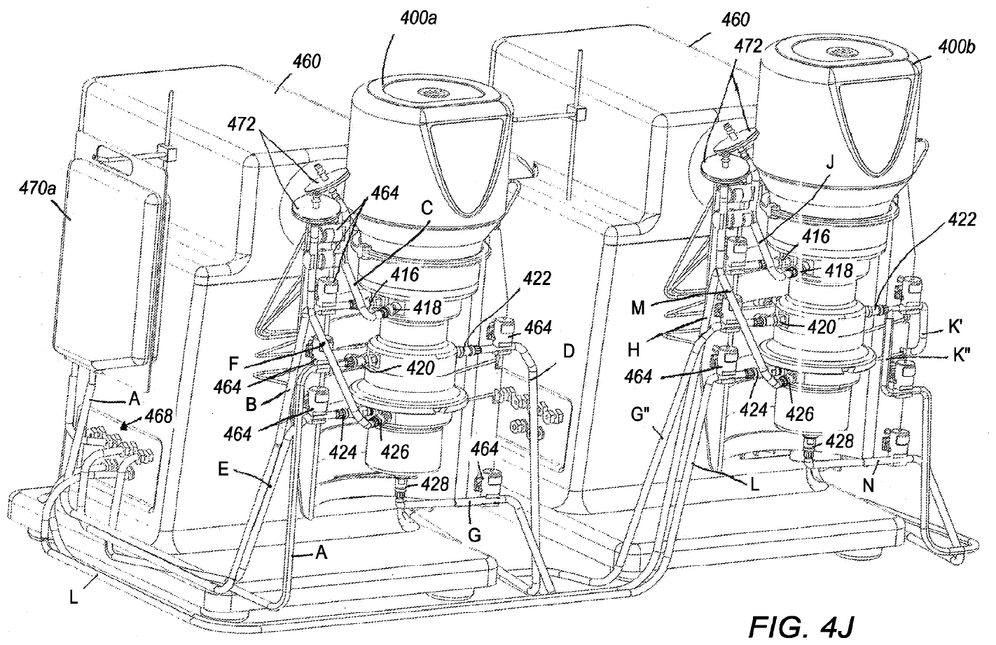

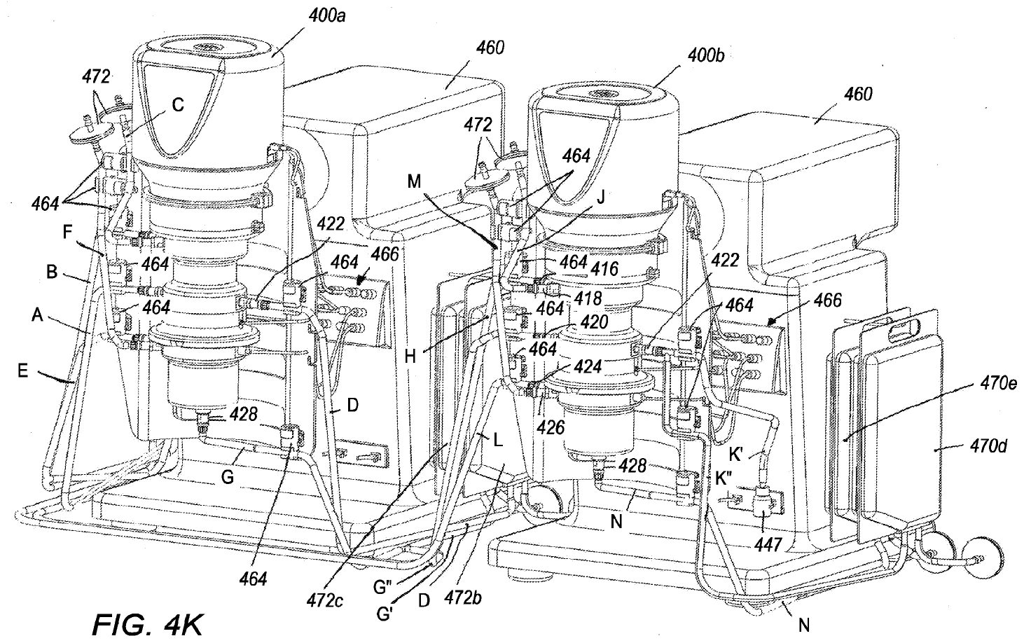

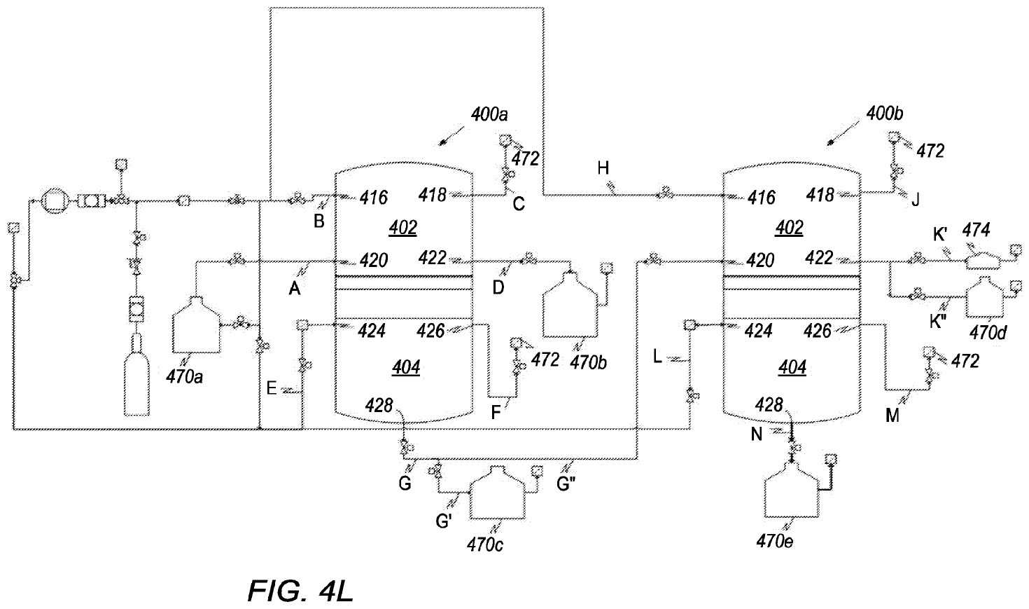

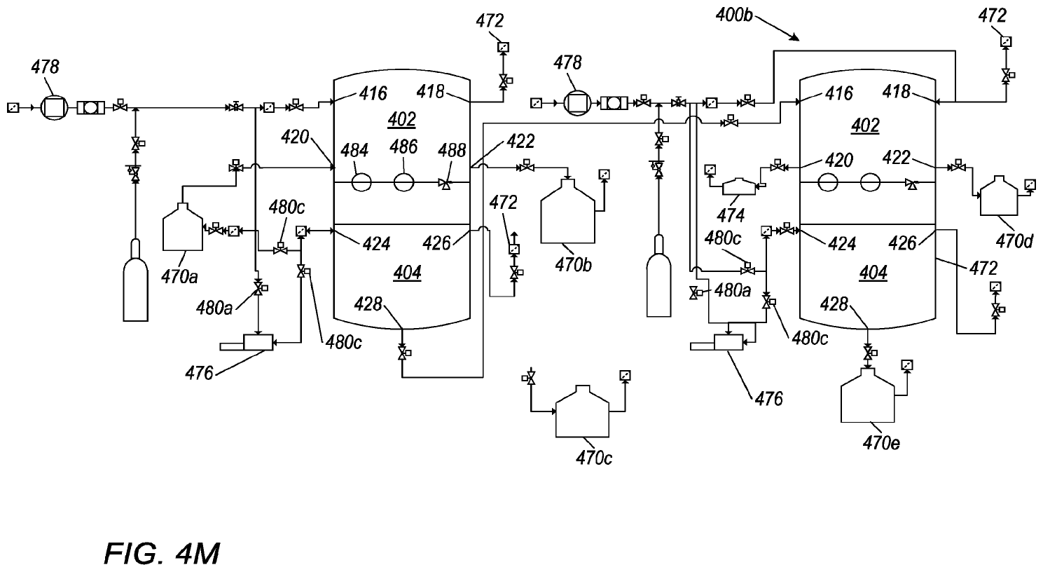

FIGS. 4A and 4B are right and left perspective views of an ultrafiltration/concentration (UC) apparatus mounted on a control tower, according to the presently disclosed subject matter;

FIG. 4C is a cross-sectional view of the UC apparatus illustrated in FIGS. 4A and 4B, taken along line V-V in FIG. 4A; and

FIG. 4D is a perspective view of a paddle unit of the UC apparatus illustrated in FIGS. 4A and 4B;

FIG. 4E is an exploded perspective cutaway view of an upper tank, stirring mechanism, and cap assembly of the UC apparatus illustrated in FIGS. 4A and 4B;

FIGS. 4F and 4G are, respectively, exploded and cutaway views of portions of the cap assembly and stirring mechanism of the UC apparatus illustrated in FIGS. 4A and 4B, according to other examples;

FIG. 4H is a perspective view of an ultrasonic shaker of the UC apparatus illustrated in FIGS. 4A and 4B;

FIGS. 4J and 4K are left and right perspective views of two of the UC apparatuses and their respective control towers, according to an example of the presently disclosed subject;

FIG. 4L is a piping and instrumentation diagram of an implementation of the arrangement illustrated in FIGS. 4J and 4K;

FIG. 4M is a piping and instrumentation diagram of implementation of an arrangement of two UC apparatuses, according to another example of the presently disclosed subject matter; and

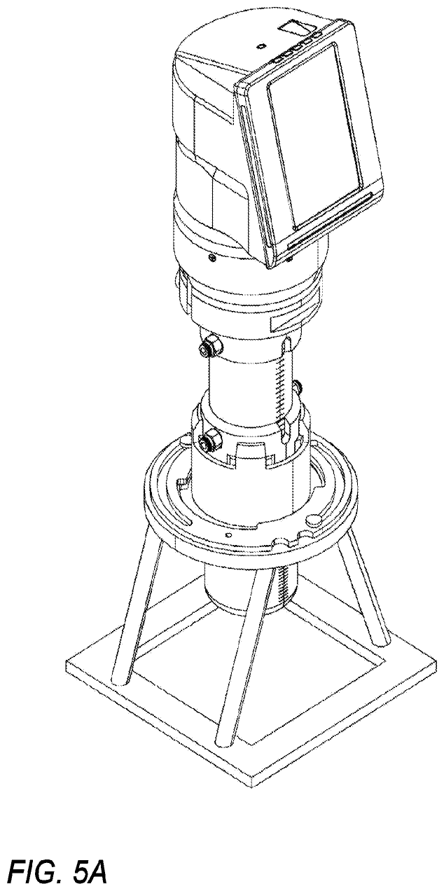

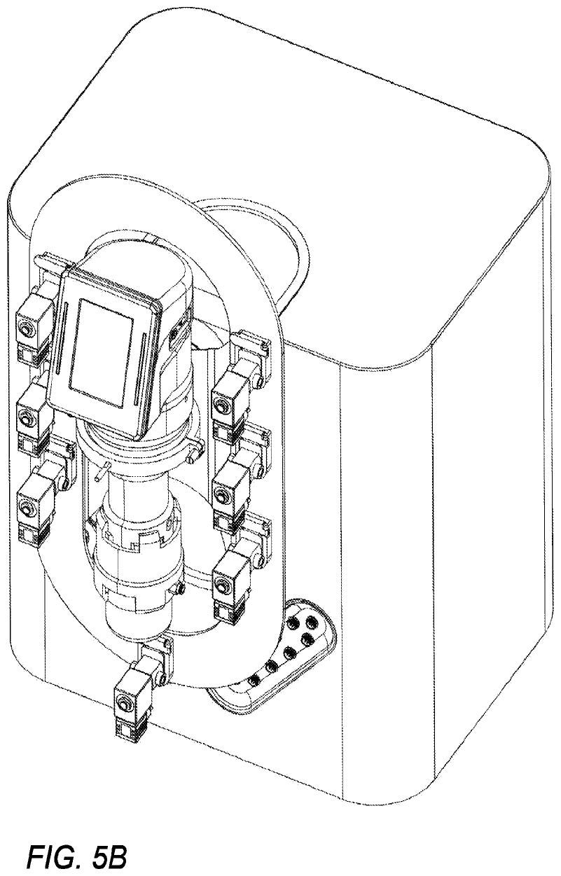

FIGS. 5A and 5B are perspective views of other examples of the UC apparatus, in a stand and mounted on a control tower, respectively.

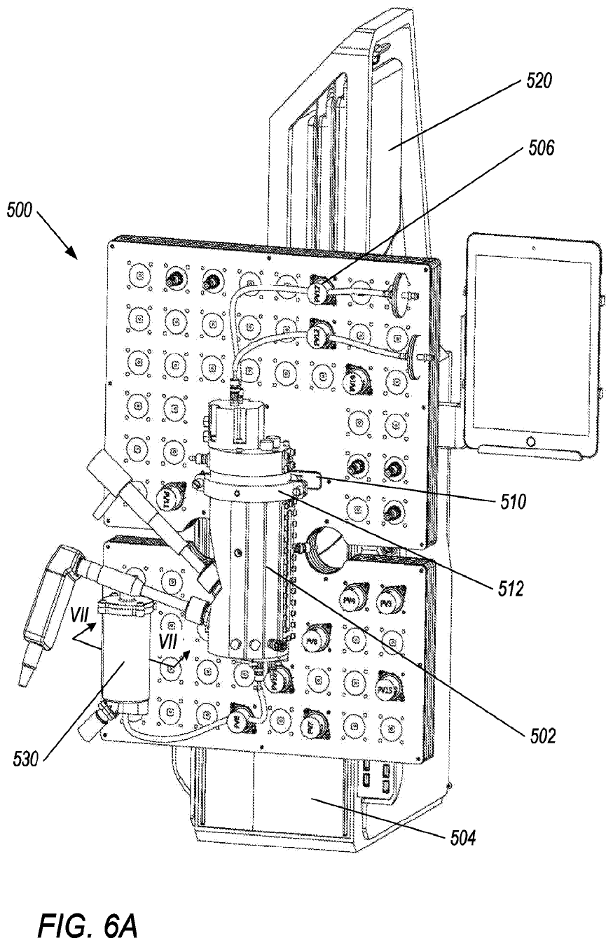

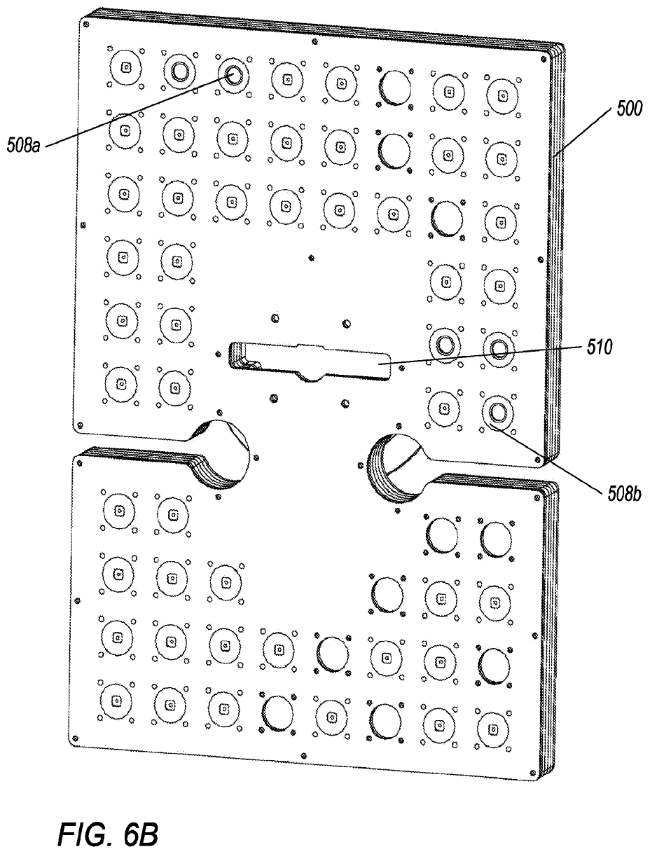

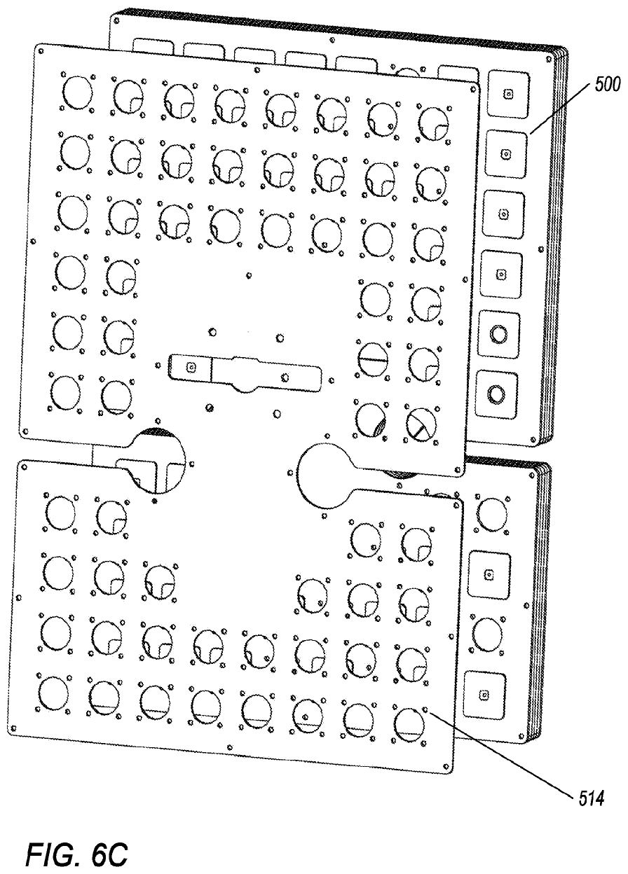

FIG. 6A is a perspective view of a connection panel according to the presently disclosed subject matter, mounted with a bioreactor on a control panel;

FIGS. 6B and 6C is a perspective view of the connection panel illustrated in FIG. 6A;

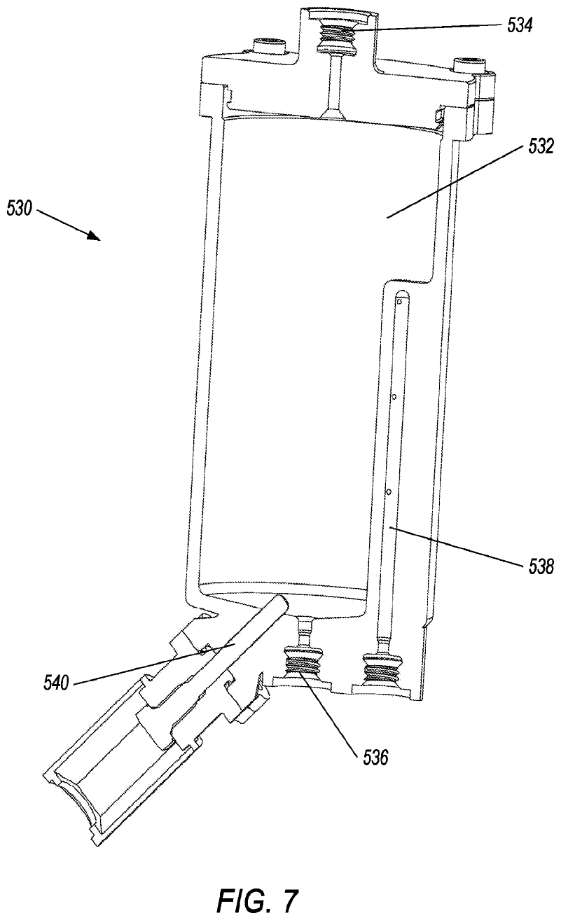

FIG. 7 is a cross-sectional view, taken along line VII-VII in FIG. 6A, of a media preparation unit according to the presently disclosed subject matter.

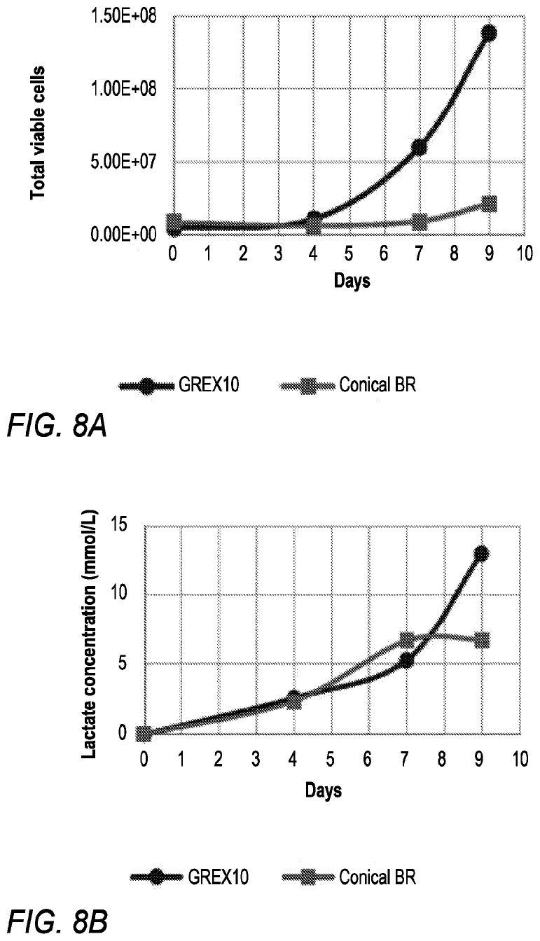

FIG. 8A shows the total viable cells measured on days 4, 7, and 9 for PBMCs expanded in the G-Rex10 and in the Conical BR.

FIG. 8B shows the lactate concentration measured on days 4, 7, and 9 for PBMCs expanded in the G-Rex10 and in the Conical BR.

It will be appreciated that for the sake of clarity, elements shown in the figures may not be drawn to scale and reference numerals may be repeated in different figures to indicate corresponding or analogous elements.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention. Further, unless otherwise required by context, singular terms shall include pluralities and plural terms shall include the singular.

In embodiments, the invention comprises a design particularly suitable for shear sensitive cell cultures, as well as other applications using disposable technologies.

Embodiments of the present invention provide various advantages over reusable bioreactors, including, without limitation, reduction of maintenance and operating costs, by eliminating, for example, operations such as preventive sterilization (SIP), cleaning (CIP), reduction in the amount of time and labor costs for regular maintenance, full compliance with validation requirements, and reducing the risk of cross-contamination.

In embodiments, the invention provides bioreactors featuring excellent control capabilities that come with disposable, reversible probes. In embodiments, the problematic galvanic and polarographic sensors, which are considered the standard in reusable systems, are replaced by disposable pO₂, pH sensors, temperature, and/or glucose, which provide the same quality level of control.

Embodiments of the present invention are suitable for use in a variety of applications, including, without limitation, tasks related to cell cultivation, shear-sensitive cell cultures, anaerobic bacteria, transition from culture flasks and shakers, cultivation of seed for traditional fermenters, production of batches in volumes up to 10 liters, scientific and industrial research, industrial developments, optimization of the production process, production of pilot lots, high-quality cultivation of seeds for industrial bioreactors, industrial production, and production of biomedical products. Embodiments of the present invention can be employed in generation of CAR-T cells and/or HEK293 cells by lentiviral transduction.

### Asymmetric Bioreactor

As illustrated in FIGS. 1A through 1G, there is provided an asymmetric bioreactor, which is generally indicated at 10, for facilitating culturing a sample of cells therewithin. The asymmetric bioreactor 10 comprises an internal cavity 12, best seen in FIGS. 1F and 1G, defined by a circumferential main housing 14 constituting a sidewall of the asymmetric bioreactor, and top and bottom covers 16, 18. A vertical axis X of the asymmetric bioreactor 10 may be defined extending perpendicularly between the top and bottom covers 16, 18.

It will be appreciated that herein the disclosure and claims, terms relating to direction, such as top, bottom, up, down, etc., and similar and/or related terms are used with reference to the orientation in the accompanying drawings based on a typical usage of the respective subject matter for clarity of description only, and are not to be construed as limiting. Moreover, the terms “vertical” and “horizontal” and similar and/or related terms are used with reference to the vertical axis X described above with reference to and as illustrated in the accompanying figures, e.g., the term “vertical” designates a direction substantially parallel to vertical axis X, and the term “horizontal” designates a designates a direction substantially perpendicular to vertical axis X, unless otherwise clear from context.

The main housing 14 may be made of any suitable material, including, but not limited to, metal (such as stainless steel), a polysulfone, a polycarbonate, or Teflon. It may be formed such that one side of an inner surface of the internal cavity 12 defined therein, e.g., a front side 20 thereof, is substantially parallel to the vertical axis X of the asymmetric bioreactor 10, i.e., it extends perpendicularly between the top and bottom covers 16, 18, while the remainder thereof is disposed at a non-parallel angle thereof. A rear side 21 of the inner surface of the internal cavity 12 may be angled with respect to the vertical axis X more than any other portion of the inner surface. Horizontal cross-sections of the cavity 12, e.g., taken in a plane perpendicular to the vertical axis X of the asymmetric bioreactor 10, may be elliptical, e.g., circular. Such cross-sections may be coaxial, for example about an axis which is angled with respect to the vertical axis X of the asymmetric bioreactor 10. Accordingly, the cavity 12 may define an asymmetrical frustoconical shape.

The asymmetric bioreactor 10 may comprise a translucent or transparent window 22, for example extending vertically along the front side 20 thereof. The window 22 may be made of any suitable material, including, but not limited to, glass, a polysulfone, or a polycarbonate, and may optionally be provided with indicia 24, for example indicating the volume of liquid within the asymmetric bioreactor.

As best seen in FIGS. 1F and 1G, the top cover 16 may comprise a main cover element 26 and an auxiliary cover element 28 mounted thereon thereabove.

The main cover element 26 is configured to be sealingly and releasingly mounted on the main housing 14. Accordingly, the main cover element 26 and main housing 14 may be formed with a mating screwing arrangement 30, for example further comprising a sealing arrangement 32, such as a suitably disposed O-ring. The main cover element 26 is formed with a through-going media bore 34, which may be disposed eccentrically therewith. In particular, a rear side 34a may be formed such that a bottom edge thereof contacts a top edge of the rear side of the inner surface of the internal cavity 12 of the main housing 14.

The auxiliary cover element 28 is configured to be sealingly and releasingly mounted on the main cover element 26. Accordingly, the main cover element 26 and auxiliary cover element 28 may be formed with a mating screwing arrangement 36, for example further comprising a sealing arrangement 38, such as a suitably disposed O-ring. The auxiliary cover element 28 is formed with a downwardly facing blind media bore 40, formed so as to align with the media bore 34 of the main cover element 26 when the auxiliary media element is mounted thereto. A suitable upper filter membrane 42 may be provided between the through-going media bore 34 of the main cover element 26 and the blind media bore 40 of the auxiliary cover element 28.

It will be appreciated that herein the specification and appended claims, references to the internal cavity 12 of the asymmetric bioreactor may include, explicitly or implicitly, the media bores 34, 40, unless otherwise clear from context or if a sensible reading of the text, for example in view of the figures, excludes such an interpretation.

The asymmetric bioreactor 10 comprises a plurality of ports, each providing fluid communication, for example selective fluid communication, between the cavity 12 and the exterior of the asymmetric bioreactor 10. Ports may be formed integrally with the main housing 14, the top cover 16, and/or the bottom cover 18, and/or may be detachably mounted thereto.

According to some examples, each of the ports comprises a socket (not indicated) formed in the main housing 14, top cover 16, or bottom cover 18, the socket allowing mounting thereto a suitable connector to facilitate fluid communication between the cavity 12 and the exterior of the asymmetric bioreactor 10. Some or all of the sockets may be similarly formed, for example having the same threading, thereby allowing mounting any of the connectors to any one of the similarly formed sockets, e.g., as determined by the manufacturer and/or a user.

The asymmetric bioreactor 10 may comprise one or more bottom ports 44, for example a first through third bottom ports 44a, 44b, 44c, disposed on a bottom surface of the bottom cover 18. The first bottom port 44a may be disposed toward the front side 20 of the asymmetric bioreactor 10, and second and third bottom ports 44b, 44c may be disposed farther away therefrom. (It will be appreciated that herein the present description, base reference numerals may be used without trailing letters to collectively refer to all elements indicated thereby; accordingly, e.g., the term “bottom port 44” may be used to collectively refer to the front bottom port 44a and the two rear bottom ports 44b, 44c, etc.)

In use, according to some examples, the first bottom port 44a may be connected to a source of suction, the second bottom port 44b may be connected to a media supply to facilitate providing media to the cavity 12 of the asymmetric bioreactor 10, and the third bottom port 44c may be connected to a gas supply, e.g., for providing pressure thereto.

The asymmetric bioreactor 10 may further comprise one or more lower-margin ports 46, for example first through fifth lower-margin ports 46a, 46b, 46c, 46d, 46e, disposed at least partially circumferentially about a lower margin of the main housing 14, i.e., adjacent the bottom cover 18.

In use, according to some examples, the first lower-margin port 46a may be used for lentivirus injection, the second lower-margin port 46b may be used for transduction agent injection, the third lower-margin port 46c may be used for injection of activators, and the fifth lower-margin port 46e may be used for seeding access. It will be appreciated that some or all of the lower-margin ports 46, similar to the fourth lower-margin port 46d of the present example, may be plugged/reserved for future use, and accordingly be closed.

The asymmetric bioreactor 10 may further comprise one or more housing-side ports 48, for example first through fourth housing-side ports 48a, 48b, 48c, 48d, for example disposed along a side of the main housing 14. It will be appreciated that the fourth (i.e., bottommost) housing-side port 48d may be disposed such that it may be considered as one of the lower-margin ports 46; its designation as one of the housing-side ports 48 is merely out of convenience, and is not to be construed as limiting in any way.

In use, according to some examples, the first and fourth housing-side ports 48a, 48d may constitute sampling access ports, and the second and third housing-side ports 48b, 48c may constitute gas supply ports. It will be appreciated that the first and second housing-side ports 48a, 48b may be used for their respective functions when the asymmetric bioreactor 10 contains relatively higher volumes (e.g., more than about 100 mL) of media, and the third and fourth housing-side ports 48c, 48d may be used for their respective functions when the asymmetric bioreactor contains relatively lower volumes (e.g., less than about 100 mL, for example about 20 mL) of media.

The asymmetric bioreactor 10 may further comprise one or more side-cover ports 50, for example first through third side-cover ports 50a, 50b, 50c, for example disposed along a side of the cover 16. According to some examples, the first and second side-cover ports 50a, 50b are disposed in the auxiliary cover element 28, thereby fluidly connecting between the blind media bore 40 and the exterior of the asymmetric bioreactor 10, and the third side-cover port 50c is disposed in the main cover element 26, thereby fluidly connecting between the through-going media bore 34 and the exterior of the asymmetric bioreactor.

In use, according to some examples, the first side-cover port 50a may be used for waste removal, and the third side-cover port 50c may be used for harvesting, for example as will be described below. The second side-cover port 50b may, depending on the protocol, be used for harvesting (e.g., in the case of an HEK293 protocol), or be plugged/reserved for future use.

The asymmetric bioreactor 10 may further comprise one or more top ports 52, for example first and second top ports 52a, 52b, disposed on a top side of the top cover 16, thereby fluidly connecting between the blind media bore 40 and the exterior of the asymmetric bioreactor 10.

In use, according to some examples, the first and second top ports 52a, 52b may be used, respectively, as a gas inlet and gas outlet port. According to some examples, each is provided with and/or connected to a check valve for this purpose.

Each of the ports may be provided according to the intended use thereof. For example, some of the ports (e.g., second lower-margin port 46b) may be configured for liquid injection. As illustrated in FIG. 1A, a port configured for liquid injection may comprise a septum assembly 54 allowing selective injection of a liquid through a seal. Each septum assembly 54 may comprise a rigid housing 56, for example made of stainless steel or any other suitable material, carrying a septum membrane 58 configured to provide fluid isolation between the cavity 12 and the exterior of the asymmetric bioreactor 10. Introduction of a liquid to the cavity 12 may be accomplished by piercing the septum membrane 58 with the needle of a syringe.

Some of the ports (e.g., second side-cover port 50b) may constitute collection ports, configured to be connected to an external collection container via a tube to facilitate collection of a product or some other portion of the contents of the asymmetric bioreactor 10. As illustrated in FIG. 1F, a collection port may comprise a threaded nipple 60. It may further comprise or be configured for connection to a tap connected thereto, to facilitate connection to a corresponding tube and to selectively bring the collection port into and out of fluid communication with the tube. The tap may be a Luer tap, and a Luer-thread adapter may be provided to facilitate connection between the nipple 60 and the tap.

Some of the ports (e.g., second housing-side port 48b) may constitute gas ports (either inlet or outlet), for example configured to be connected to an external gas source, e.g., a tank, and/or a source of suction via a tube, to facilitate provisioning/removal of a gas to/from the asymmetric bioreactor 10. According to some examples, one or more of the gas ports may be in communication with the external environment, e.g., to facilitate venting. As illustrated in FIG. 1G, a gas port may comprise a threaded nipple 62. It may further comprise or be configured for connection to a tap connected thereto, to facilitate connection to a corresponding tube and to selectively bring the gas port into and out of fluid communication with the tube. The tap may be a Luer tap, and a Luer-thread adapter may be provided to facilitate connection between the nipple 62 and the tap. An air filter may be provided between the tap and the tube. According to some examples, one or more of the gas ports may each comprise or be connected to a check valve. According to some examples, one or more of the gas ports may each comprise or be connected to a gas filter.

Some of the ports (e.g., first housing-side port 48a) may constitute needleless access ports, each of which is configured to allow controlled introduction and/or extraction of material therethrough between the cavity 12 and the exterior of the asymmetric bioreactor 10. Each of the needleless access ports may comprise or be configured for connection to a swabable valve, configured for selectively being filled with, holding, and releasing a controlled amount of fluid. Some or each of the needleless access ports may comprise one of the sockets with a swabable valve connected thereto. According to some examples, some or each of the sockets may be threaded, with the swabable valve connected directly thereto. According to other examples, some or each of the sockets may be threaded, with the swabable valve being connected thereto via an adapter, e.g., the swabable valve may comprise a Luer connection and be connected to its respective socket via a Luer-thread adapter.

Some of the ports (e.g., second bottom port 44b) may constitute liquid supply ports, each of which is configured to be connected to an external supply container via a tube, to facilitate providing a liquid to the cavity 12 of the asymmetric bioreactor 10. Each of the liquid supply ports may comprise or be configured for connection to a check valve, configured for allowing passage of a fluid therethrough only in a direction towards the cavity 12. Some or each of the liquid supply ports may comprise one of the sockets with a check valve connected thereto. According to some examples, some or each of the sockets may be threaded, with the check valve connected directly thereto. According to other examples, some or each of the sockets may be threaded, with the check valve being connected thereto via an adapter, e.g., the check valve may comprise a Luer connection and be connected to its respective socket via a Luer-thread adapter.

It will be appreciated that the uses of the valves mentioned above is by way one non-limiting example only, and that one having skill in the art may realize other uses for some or all of the valves, for example as suited for the process for which the asymmetric bioreactor 10 is being used. It will be further appreciated that, as mentioned above, each of the ports comprises a socket which facilitates mounting thereto a suitable connector to facilitate fluid communication, each of the ports may be easily adapted for one of several different kinds of uses, for example by the manufacturer or by the user.

The asymmetric bioreactor 10 may comprise and/or be configured to accommodate one or more probes, such that one end (e.g., a sensing end) of the probe is disposed within the cavity 12, and an opposite end of the probe is disposed externally thereto. The probes may be of any suitable type. For example, they may comprise a pO₂ (i.e., dissolved oxygen) sensor, a pH sensor, a temperature sensor, a glucose sensor, a pressure sensor, a biomass sensor, and/or a lactate sensor. According to some examples, at least one of the probes is a heating probe, configured to be inserted into the cavity 12 to heat the media therewithin. Some or all of the sensors may be single-use and/or reversible. It will be appreciated that a combination of some or all of the sensors provided may obviate the need to provide a galvanic sensor or a polarographic sensor, for example depending on the intended use of the asymmetric bioreactor 10.

Accordingly, the asymmetric bioreactor 10 may comprise one or more lower probe ports 64, disposed near a bottom end thereof. Each of the lower probe ports 64 is configured to securely and sealingly hold a probe or plug (in the event that a probe is not used) therewithin. Each lower probe port 64 may comprise a collet 66 attached to and projecting outwardly from an exterior side of the main housing 14 of the asymmetric bioreactor 10, and a corresponding nut 68 threadingly mountable thereto to tightly close the collet over an inserted probe or plug (commonly indicated at 70). When the collet 66 is so closed over the probe, a sensing end there of is disposed within the cavity 12, and an opposite end thereof, which for example may be connectable to a computer or other suitable device to obtain data from the probe and/or to control it, projects outwardly from the main housing 14.

The asymmetric bioreactor 10 may further comprise one or more upper probe ports 72, disposed near a top end thereof, for example in the top cover 16. Each of the upper probe ports 72 may comprise a through-going bore formed substantially vertically through the top cover 16, with one or more sealing arrangements 74, for example O-rings, provided therein. A probe or plug 70 may be provided in each of the upper probe ports 56, disposed therewithin similarly as described above with reference to the lower probe ports 48, mutatis mutandis.

At least some of the upper probe ports 72 may be formed within the main cover element 26.

According to some examples, the third side-cover port 50c is formed in the main cover element 26, for example in the rear side 34a of the through-going media bore 34, e.g., below the upper filter membrane 42, and the first and second side-cover ports 50a, 50b are formed in the auxiliary cover element 28, and are open to the blind media bore 40, i.e., above the upper filter membrane 42.

As mentioned, the third side-cover port 50c may be used for harvesting. Its location may facilitate control of the quantity of harvest material collected, in particular facilitating collection of small quantities thereof. For example, as illustrated in FIG. 1H, the asymmetric bioreactor 10 may be tilted until a liquid containing harvest material therewithin just reaches the corner formed between the rear side 21 of the internal cavity 12 and the rear side 34a of the through-going media bore 34. In this position, the rear side 34a of the through-going media bore 34 slopes upwardly toward the third side-cover port 50c, thereby preventing the liquid from progressing further theretoward. The asymmetric bioreactor 10 may be further rotated in a controlled manner such that a predetermined, e.g., small, quantity of liquid flows toward the third side-cover port 50c for collection therethrough.

In addition, the location of the third side-cover port 50c may facilitate collection of substantially all of the harvest material from within the internal cavity 12. For example, during collection, the asymmetric bioreactor 10 may be tilted such that all of the liquid therein flows toward the third side-cover port 50c. Owing to its location adjacent the upper filter membrane 42, harvest material which is blocked thereby from flowing into the blind media bore 40 pools in the area directly adjacent the third side-cover port 50c, and may thus be substantially completely collected.

The bottom cover 18 is configured to be sealingly and releasingly mounted on the main housing 14. Accordingly, the bottom cover 18 and main housing 14 may be formed with a mating screwing arrangement 76, for example further comprising a sealing arrangement 78, such as a suitably disposed O-ring. A suitable bottom filter membrane 80 may be provided between the bottom cover 18 and the main housing 14, for example above the bottom ports 44.

In use, gas may accumulate between the bottom cover 18 and the bottom filter membrane 80, which may impede and/or prevent media supplied, for example via the second bottom port 44b, from reaching the internal cavity 12. Accordingly, for example as best seen in FIG. 1J, the first bottom port 44a may be disposed such that it projects above the upper surface of the bottom cover 18, thereby minimizing the distance between it and the bottom filter membrane 80. Accordingly, any gas which may accumulate directly below the bottom filter membrane 80 may be suctioned out via the first bottom port 44a. This may mitigate the effect of the accumulated gas, thereby facilitating a more effective passage of media into the internal cavity 12.

The asymmetric bioreactor 10 may be formed with a collar 82 defining a circumferential groove 84 therebelow. According to some examples, the collar 82 and groove 84 are both formed in the main housing 14 as illustrated. According to other examples, the collar 82 and groove 84 are both formed in the top cover 16. According to further examples, the top cover 16, when mounted on the main housing 14 for example as described above, constitutes the collar 82, and a gap formed therebelow constitutes the groove 84.

As illustrated in FIG. 1K, the asymmetric bioreactor 10 may comprise a heating panel 86, configured to be wrapped around the main housing 14 to facilitate maintaining a predetermined temperature therein. The heating panel is formed with a plurality of cutouts 88 to facilitate being mounted directly on the outer surface of the main housing 14. For example, it may comprise a window cutout 88a configured to lie in registration over the window 22, and a plurality of port cutouts 88b, each configured to lie in registration over one of the ports formed in the asymmetric bioreactor 10, for example as described above. A plurality of solder pads 90 may be provided to facilitate connecting the heating panel 86 to an electrical circuit, e.g., to facilitate control thereof. Clamps (not illustrated) may be provided to secure the heating panel 86 to the main housing 14.

As illustrated in FIG. 1L, the asymmetric bioreactor 10 may be provided as part of a system which comprises, inter alia, the asymmetric bioreactor and a control tower 92. The control tower 92 comprises a support arm 94 configured to be received within the groove 84 of the asymmetric bioreactor 10 for secure holding thereof. The control tower 92 may comprise an actuator (not illustrated), for example comprising a stepper motor, configured to selectively pivot the asymmetric bioreactor 10 to one or more tilt angles during use. The control tower 92 may be further configured to regulate the flow of fluids (e.g., gases, media, harvested material, etc.) in and out of the asymmetric bioreactor 10.

In embodiments, the system comprising the asymmetric bioreactor 10 and control tower 92 operates by swinging or tilting the supported bioreactor, and the design may include, inter alia, flexible and adoptable step motor controllable settings such as tilting speed and inclining motion routes. This disposable bioreactor is scalable with a working range of 15 ml-10 liters, and can include disposable sensors, such as pO₂, pH, temperature, and/or glucose or other sensors.

Generally, in operation, cells are seeded, and media is introduced through appropriate ports into the reactor vessel. Activation and transduction involve soft tilting (e.g., approximately 5-15°/minute), with a maximum tilt angle being determined by volume. The maximum tilt angle can range from about 23° from vertical axis to about 83° from vertical axis. In embodiments, maximum tilt angle is about 23° from vertical axis when the bioreactor is filled with about 15 mL. In embodiments, maximum tilt angle is about 61° from vertical axis when the bioreactor is filled with about 100 mL. In embodiments, maximum tilt angle is about 73° from vertical axis when the bioreactor is filled with about 300 mL. In embodiments, maximum tilt angle is about 83° from vertical axis when the bioreactor is filled with about 500 mL. In embodiments, the bioreactor is tilted to horizontal position for performing harvest and waste removal steps and back to vertical position for introduction of transduction agents, transfection mix, etc. Wash steps, introduction of fresh media, and other steps are performed as appropriate, and as further described herein.

As illustrated in FIG. 2, the asymmetric bioreactor 10 may be associated with various peripheral devices to facilitate and/or achieve performance of its intended function. In the illustrated example, the system scheme 200 includes asymmetric bioreactor 10, as described above with reference to and as illustrated in one or more of FIGS. 1A through 1L. The system scheme 200 as illustrated further includes a first harvest reservoir 201a for containing harvest from a CAR-T process via the third side-cover port 50c, waste reservoir 201b for containing waste via the second side-cover port 50b, and a second harvest reservoir 201c for containing harvest from an HEK293 process via the first side-cover port 50a, as well as media reservoir 201d for providing media to the asymmetric bioreactor 10 via the second bottom port 44b, and washing buffer reservoir 201e for providing washing buffer to asymmetric bioreactor 10 via the second bottom port 44b. Reservoirs 201a-201e can be any suitable container for performing their intended functions, including, without limitation and in any combination, bags, bottles, Labtainers, or the like.

The Examples provide further details of processes, their steps, order of operation, and other protocol aspects for which the asymmetric bioreactor 10, other bioreactors, and/or peripheral devices described herein are suitable.

In embodiments, cells can be seeded through the fifth lower-margin port 46e, which seeding can be facilitated by use of appropriate components described herein, such as, e.g. luer lock syringe 209, swabable valve 208, adapter thread to luer 212, and/or others. Media can flow from Labtainer 201d through dual channel peristaltic pump 203 and oxygenator 204 and then introduced via the second bottom port 44b. Oxygenator 204 is configured also to receive oxygen from oxygen gas balloon 202a to oxygenate the media. Mass flow controller 205a can be employed to facilitate this aspect of the process. Gas can be introduced into the asymmetric bioreactor 10 from one or more of compressed air balloon 202b, nitrogen balloon 202c, or carbon dioxide balloon 202d. The appropriate gas can be flowed through static mixer 213, facilitated by a mass controller 205 associated with the particular gas balloon. The gas can enter the asymmetric bioreactor 10 via any of the second top port 52b, the second or third housing-side ports 48b, 48c, or the third bottom port 44c, as described herein. Gas can be extracted or released as appropriate via the first top port 52a.

Activators can be introduced through the third lower-margin port 46c, via a syringe and/or luer lock needle 210.

Lentivirus can be introduced into the first lower-margin port 46a via a syringe and/or luer lock needle 210 or other suitable mechanism. Transduction agent can be introduced into the second lower-margin port 46b via a syringe and/or luer lock needle 210 or other suitable mechanism. The bioreactor can be filled with formulation buffer through the fourth lower-margin port 46d via a syringe and/or luer lock needle 210 or other suitable mechanism. Samples can be taken through the first and/or fourth housing-side port 48a, 48d. In embodiments, the sample can be extracted through the ports, via, inter alia, adapter thread to luer 212, swabable valve 208, luer lock syringe 209, and/or any other suitable components.

### Cylindrical Bioreactor

As illustrated in FIGS. 3A through 3C, there is provided a cylindrical bioreactor, which is generally indicated at 300, for facilitating culturing a sample of cells therewithin. The cylindrical bioreactor 300 comprises an internal cavity, defined by a circumferential main housing 314 constituting a sidewall of the cylindrical bioreactor, and top and bottom covers 316, 318. A vertical axis X of the cylindrical bioreactor 300 may be defined extending perpendicularly between the top and bottom covers 316, 318.

The main housing 314 may be made of any suitable material. According to some examples, the main housing 314 is transparent, for example being made of one or more materials selected from a group including, but not limited to, glass, a polysulfone, or a polycarbonate.

The cylindrical bioreactor 300 comprises a plurality of ports, each providing fluid communication, for example selective fluid communication, between the interior and the exterior of the cylindrical bioreactor. Ports may be formed integrally with the main housing 314, the top cover 316, and/or the bottom cover 318, and/or may be detachably mounted thereto.

According to some examples, the main housing 314, top cover 316, and/or bottom cover 318 may be formed with a plurality of sockets (not indicated), each allowing mounting thereto of a suitable connector to the facilitate fluid communication between the internal cavity and the exterior of the cylindrical bioreactor 300. Some or all of the sockets may be similarly formed, for example having the same threading, thereby allowing mounting any of the connectors to any one of the similarly formed sockets, e.g., as determined by the manufacturer and/or a user.

Each of the ports may be provided according to the intended use thereof. For example, the cylindrical bioreactor 300 may comprise one or more liquid injection ports 326, each of which may be provided similarly to the liquid injection ports 26 of the asymmetric bioreactor 10 described above with reference to and as illustrated in one or more of FIGS. 1A through 1L. For example, each of the liquid injection ports 326 of the cylindrical bioreactor 300 may comprise a septum assembly allowing selective injection of a liquid through a seal. Each septum assembly may comprise a rigid housing, for example made of stainless steel or any other suitable material, carrying a septum membrane configured to provide fluid isolation between the internal cavity and the exterior of the cylindrical bioreactor 300. Introduction of a liquid to the internal cavity of the cylindrical bioreactor 300 may be accomplished by piercing the septum membrane with the needle of a syringe.

According to some examples, the liquid injection ports may comprise some or all of a formulation buffer injection port 326a, a lentivirus injection port 326b, a transduction agent injection port 326c, and an activators injection port 326d.

The cylindrical bioreactor 300 may further comprise one or more collection ports 334, each of which is configured to be connected to an external collection container via a tube, to facilitate collection of a product or some other portion of the contents of the cylindrical bioreactor. Each of the collection ports 334 of the cylindrical bioreactor 300 may be provided similarly to the collection ports 34 of the asymmetric bioreactor 10 described above with reference to and as illustrated in one or more of FIGS. 1A through 1L. For example, each of the collection ports 334 of the cylindrical bioreactor 300 may comprise a threaded nipple, e.g., connected to the top cover 316. It may further comprise or be configured for connection to a tap connected thereto, to facilitate connection to a corresponding tube and to selectively bring the collection port 334 into and out of fluid communication with the tube. The tap may be a Luer tap, and a Luer-thread adapter may be provided to facilitate connection between the nipple and the tap.

According to some examples, the collection ports 334 may comprise some or all of a first harvest collection port 334a, e.g., configured for collection of chimeric antigen receptor T-cells, a waste collection port 334b, and a second harvest collection port 334c.

The cylindrical bioreactor 300 may further comprise one or more gas supply ports 338, each of which is configured to be connected to an external gas source, e.g., a tank, via a tube, to facilitate provisioning of a gas to the cylindrical bioreactor. Each of the gas supply ports 338 of the cylindrical bioreactor 300 may be provided similarly to the gas supply ports 38 of the asymmetric bioreactor 10 described above with reference to and as illustrated in one or more of FIGS. 1A through 1L. For example, each of the gas supply ports 338 of the cylindrical bioreactor 300 may comprise a threaded nipple, e.g., connected to the main housing 314 or to the top cover 316. It may further comprise or be configured for connection to a tap connected thereto, to facilitate connection to a corresponding tube and to selectively bring the gas supply port 338 into and out of fluid communication with the tube. The tap may be a Luer tap, and a Luer-thread adapter may be provided to facilitate connection between the nipple 40 and the tap. An air filter may be provided between the tap and the tube.

According to some examples, the gas supply ports 338 may comprise some or all of a 100 mL gas supply port 338a (e.g., for introduction of gas to 100 mL of media), a 20 mL gas supply port 338b (e.g., for introduction of gas to 20 mL of media), and an option gas supply port 338c.

The cylindrical bioreactor 300 may further comprise one or more needleless access ports 342, each of which is configured to allow controlled introduction and/or extraction of material therethrough between the internal cavity and the exterior of the cylindrical bioreactor. Each of the needleless access ports 342 of the cylindrical bioreactor 300 may be provided similarly to the needleless access ports 42 of the asymmetric bioreactor 10 described above with reference to and as illustrated in one or more of FIGS. 1A through 1L. For example, each of the needleless access ports 342 of the cylindrical bioreactor 300 may comprise or be configured for connection to a swabable valve, configured for selectively being filled with, holding, and releasing a controlled amount of fluid. Some or each of the needleless access ports 342 may comprise one of the sockets with a swabable valve connected thereto. According to some examples, some or each of the sockets may be threaded, with the swabable valve connected directly thereto. According to other examples, some or each of the sockets may be threaded, with the swabable valve being connected thereto via an adapter, e.g., the swabable valve may comprise a Luer connection and be connected to its respective socket via a Luer-thread adapter.

According to some examples, the needleless access ports 342 may comprise some or all of a first sampling access port 342a, a second sampling access port 342b, and a seeding access port 342c.

The cylindrical bioreactor 300 may further comprise one or more liquid supply ports 344, each of which is configured to be connected to an external supply container via a tube, to facilitate providing a liquid to the internal cavity of the cylindrical bioreactor. Each of the liquid supply ports 344 of the cylindrical bioreactor 300 may be provided similarly to the liquid supply ports 44 of the asymmetric bioreactor 10 described above with reference to and as illustrated in one or more of FIGS. 1A through 1L. For example, each of the needleless access ports 342 of the cylindrical bioreactor 300 may comprise or be configured for connection to a check valve, configured for allowing passage of a fluid therethrough only in a direction towards the internal cavity of the cylindrical bioreactor 300. Some or each of the liquid supply ports 344 may comprise one of the sockets with a check valve connected thereto. According to some examples, some or each of the sockets may be threaded, with the check valve connected directly thereto. According to other examples, some or each of the sockets may be threaded, with the check valve being connected thereto via an adapter, e.g., the check valve may comprise a Luer connection and be connected to its respective socket via a Luer-thread adapter.

According to some examples, the liquid supply ports 344 may comprise some or both of a washing buffer supply port 344a and a media supply port 344b.

The cylindrical bioreactor 300 may further comprise one or more upper gas ports 346, for facilitating provision and removal of gas from an upper portion of the internal cavity. Each of the upper gas ports 346 of the cylindrical bioreactor 300 may be provided similarly to the upper gas ports 46 of the asymmetric bioreactor 10 described above with reference to and as illustrated in one or more of FIGS. 1A through 1L. For example, each of the upper gas ports 346 of the cylindrical bioreactor 300 may comprise or be configured for connection to a check valve, configured for allowing passage of gas therethrough only in a single direction. Some or each of the upper gas ports 346 may comprise one of the sockets with a check valve connected thereto. According to some examples, some or each of the sockets may be threaded, with the check valve connected directly thereto. According to other examples, some or each of the sockets may be threaded, with the check valve being connected thereto via an adapter, e.g., the check valve may comprise a Luer connection and be connected to its respective socket via a Luer-thread adapter.

Each of the upper gas ports 346 may further comprise an air filter.

According to some examples, the upper gas ports 346 may comprise some or both of an upper gas inlet port 346a, in which the check valve is configured to allow gas to flow only in a direction toward the internal cavity of the cylindrical bioreactor 300, and an upper gas outlet port 346b, in which the check valve is configured to allow gas to flow only in a direction away from the internal cavity. The gas inlet port 346a may be connected to, or configured for connection to, a tube connected to a gas source. The gas outlet port 346b may be in direct fluid communication with the environment, for example via a gas filter.

It will be appreciated that while intended uses of the ports has been described above, in practice each may be used for any suitable purpose, mutatis mutandis. According to some examples, one of the ports provided near the bottom of the main housing 314 may be configured for harvest collection, for example in the location disclosed herein for the transduction agent injection port 326c. As seen, e.g., in FIG. 3D, below this port location is a frustoconical section. Accordingly, and similar to as described above with reference to and as illustrated in FIG. 1H, when the cylindrical bioreactor 300 contains a small amount of liquid containing harvest material, it may be tilted so as to collect the liquid in the corner formed between the top of the frustoconical portion and the vertical wall; further controlled tilting may be employed to bring a small quantity of liquid to the port for collection therethrough.

In addition, and further similar to as described above with reference to and as illustrated in FIG. 1H, this arrangement may facilitate collection of substantially all of the harvest material from within the cylindrical bioreactor 300, mutatis mutandis.

The cylindrical bioreactor 300 may comprise and/or be configured to accommodate one or more probes, such that one end (e.g., a sensing end) of the probe is disposed within the internal cavity, and an opposite end of the probe is disposed externally thereto. The probes may be of any suitable type. For example, they may comprise a pO₂ (i.e., dissolved oxygen) sensor 345, a CO₂ sensor 347, a biomass sensor 349, and/or a glucose/lactate sensor 351. According to some examples, at least one of the probes is a heating probe, configured to be inserted into the internal cavity to heat the media therewithin.

Some or all of the sensors may be single-use and/or reversible. It will be appreciated that a combination of some or all of the sensors provided may obviate the need to provide a galvanic sensor or a polarographic sensor, for example depending on the intended use of the cylindrical bioreactor 300.

Accordingly, the cylindrical bioreactor 300 may comprise one or more lower probe ports 348, disposed near a bottom end thereof. Each of the lower probe ports 348 is configured to securely and sealingly hold a probe or plug (in the event that a probe is not used) therewithin. Each of the lower probe ports 348 of the cylindrical bioreactor 300 may be provided similarly to the lower probe ports 348 of the asymmetric bioreactor 10 described above with reference to and as illustrated in one or more of FIGS. 1A through 1L. For example, each lower probe port 348 may comprise a collet attached to and projecting outwardly from an exterior side of the main housing 314 of the cylindrical bioreactor 300, and a corresponding nut threadingly mountable thereto to tightly close the collet over an inserted probe or plug. When the collet is so closed over the probe, a sensing end there of is disposed within the internal cavity of the cylindrical bioreactor 300, and an opposite end thereof, which for example may be connectable to a computer or other suitable device to obtain data from the probe and/or to control it, projects outwardly from the main housing 314.

As illustrated in FIG. 3D, the top cover 316 may comprise a main cover element 358 and an auxiliary cover element 360 mounted thereon thereabove.

The main cover element 358 is configured to be sealingly and releasingly mounted on the main housing 314. Accordingly, the main cover element 358 and main housing 314 may be formed with a mating screwing arrangement 362, for example further comprising a sealing arrangement 364, such as a suitably disposed O-ring. The main cover element 358 defines a through-going media bore 366.

The auxiliary cover element 360 is configured to be sealingly and releasingly mounted on the main housing 314 of the cylindrical bioreactor 300. Accordingly, the main cover element 358 and auxiliary cover element 360 may be formed with a mating screwing arrangement 368, for example further comprising a sealing arrangement 370, such as a suitably disposed O-ring. The auxiliary cover element 360 is formed with a downwardly facing blind media bore 372, formed so as to align with the media bore 366 of the main cover element 358 when the auxiliary media element is mounted thereto. A suitable upper filter membrane 374 may be provided between the through-going media bore 366 of the main cover element 358 and the blind media bore 372 of the auxiliary cover element 360.

According to some examples, the first harvest collection port 334a, waste collection port 334b, upper gas inlet port 346a, and upper gas outlet port 346b are formed in the auxiliary cover element 360, and are open to the blind media bore 372, i.e., above the upper filter membrane 374.

The bottom cover 318 is configured to be sealingly and releasingly mounted on the main housing 314. Accordingly, the bottom cover 318 and main housing 314 may be formed with a mating screwing arrangement 376, for example further comprising a sealing arrangement 378, such as a suitably disposed O-ring. A suitable bottom filter membrane 380 may be provided between the bottom cover 318 and the main housing 314.

Some of the ports, for example the washing buffer supply port 344a, media supply port 344b, and option gas supply port 338c, may be formed in the bottom cover 318 below the bottom filter membrane 380.

It will be appreciated that a cylindrical bioreactor may be provided with different numbers of ports and sensors, for example as illustrated in FIG. 3E.

### Ultrafiltration/Concentration System

As illustrated in FIGS. 4A through 4C, there is provided an ultrafiltration/concentration (UC) apparatus, which is generally indicated at 400, for facilitating filtering and/or concentrating a sample of cells therewithin. The UC apparatus 400 comprises upper and lower tanks 402, 404 connected by a bridging unit 406, and a stirring mechanism 408. A flow passage 410 is provided in the bridging unit 406, providing fluid connection between the upper and lower tanks 402, 404. It further comprises a processor (not illustrated) configured to direct operation thereof.

The UC apparatus 400 further comprises, or is configured to be provided with, a suitable filter support 412 in which the flow passage 410 is formed, and which is disposed such that liquid in the upper tank 402 must pass therethrough before entering the lower tank 404 via the flow passage. According to some examples, the filter support 412 is disposed at a lower open side of the upper tank 402.

The UC apparatus 400 comprises a plurality of ports, each providing fluid communication, for example selective fluid communication, between the interior of the UC apparatus and the exterior thereof. Ports may be formed integrally with other elements (e.g., tanks 402, 404; bridging unit 406) of the UC apparatus 400, and/or detachably mounted thereto.

According to some examples, the UC apparatus 400 may be formed with a plurality of sockets 414, each allowing mounting thereto of a suitable connector to the facilitate fluid communication between the interior and exterior of the UC apparatus. Some or all of the sockets may be similarly formed, for example having the same threading, thereby allowing mounting any of the connectors to any one of the similarly formed sockets, e.g., as determined by the manufacturer and/or a user.

The upper tank 402 may comprise a first upper-top port 416 and a second upper-top port 418 disposed near a top end thereof, and first and second upper-bottom ports 420, 422 disposed near a bottom end thereof. The lower tank 404 may comprise first and second lower-top ports 424, 426 disposed near a top end thereof, and lower-bottom port 428 disposed near a bottom end thereof the lower tank, for example in a bottommost surface thereof.

The stirring mechanism 408 is configured to selectively mix the contents of the upper tank 402. According to some examples, it may comprise a magnetic stirrer comprising a paddle unit 430 within the upper tank 402, the paddle unit having one or more vertically disposed paddles 432 attached to a vertical support 434 which extends upwardly along a vertical axis X of the UC apparatus 400, and disposed directly above and adjacent the filter support 412.

As illustrated in FIG. 4D, each of the paddles 432 may be shaped so as to facilitate moving residue in order to prevent it from accumulating on the filter therebelow, and in particular from accumulating in the center thereof above the flow passage 410. Moreover, they may be designed to prevent and/or reduce cavitation. According to some examples, each of the paddles 432 may extend outwardly from the vertical axis X along a straight line, thereby providing stirring while allowing particles to be moved outwardly under a centrifugal force. In addition, each of the paddles 432 may be angled upwardly and away from the filter in the direction of rotation, such that during rotation, it applies and upward bias away from the filter.

The vertical support 434 extends upwardly through the upper tank 402 and is fixedly received within a rotor unit 436 disposed thereabove. A cap assembly 438 is provided, comprising a track 440 formed therewithin in which the rotor unit 436 is received.

As seen in FIG. 4E, the rotor unit 436 according to some examples may comprise a plurality of permanent magnets 442 therewithin. The cap assembly 438 may be configured to produce a magnetic field with reversing polarity, e.g., comprising an armature (not illustrated), thereby inducing the permanent magnets 442 and thus to rotor unit 436 and the paddle unit 430 attached thereto to rotate about a vertical axis X of the UC apparatus 400. As the rotation is induced by magnetic interaction between the stirring mechanism 408 and a drive mechanism (e.g., the armature) which is within the cap assembly 438, fluid isolation may be maintained between the interior of the upper tank 402 and the drive mechanism, thereby eliminating the need to provide seals which may be subject to failure, e.g., leaks, during use of the UC apparatus 400, which otherwise could compromise the sterility of its contents.

According to other examples, for example as illustrated in FIGS. 4F and 4G, the rotor unit 436 comprises a plurality of electromagnets and is rotatably received with a cover 435 which closes the top of the upper tank 402. The vertical support 434 of the paddle unit 430 is attached to the rotor 436, such that it rotates therewith. A stationary stator 437, comprising a plurality of electromagnets configured to produce a rotating magnetic field, is disposed about the cover 435. The polarities of the electromagnets are varied as is well-known in the art to induce rotation of the rotor unit 436.

The stirring mechanism 408 may further comprise a support element 444 configured to facilitate stabilization of the paddle unit 430. The support element 434 may comprise a through-going aperture 446 through which the paddle unit 430 passes, and two or more support arms 448 secured to a non-rotating element of the UC apparatus 400, e.g., to an inner wall of the upper tank 402.

The UC apparatus 400 may further comprise an ultrasonic shaker, which is generally indicated at 450. The ultrasonic shaker is configured to vibrate at an ultrasonic frequency, e.g., in the rage of about 20 kHz to about 40 kHz, in order to dislodge residue from the filter. Accordingly, it may be provided directly below the filter support 412 (as illustrated), integrated therewith (not illustrated), or in any other suitable manner. It will be appreciated that while herein the specification and appended claims and ultrasonic shaker is described, a shake which vibrates at any other suitable frequency may be provided without departing from the scope of the presently disclosed subject matter, mutatis mutandis.

As illustrated in FIG. 4H, the ultrasonic shaker 450 may comprise one or more piezoelectric elements 452 and an electrical connection 454. In use, a variable electrical signal having a desired frequency is applied via the electrical connection 454, causing the piezoelectric elements to vibrate at a corresponding frequency, as per the inverse piezoelectric effect. Alternatively or in addition thereto, the ultrasonic shaker 450 may be configured to receive, via the electrical connection 454, a non-varying current and/or a current varying at a frequency different from a desired one, and to produce a variable electrical signal of a desired frequency and apply it to the piezoelectric elements, thereby causing them to vibrate at a corresponding frequency.

The UC apparatus 400 further comprises one or more sensors, not illustrated, for example in communication with the controller to provide information regarding measured data thereto. According to some examples, the sensors include one or more selected from the group including a pressure/vacuum sensor, a temperature sensor (internal and/or external), a media level sensor, a pH sensor, a Hall-effect sensor (e.g., to measure the rate of rotation of the stirring mechanism 408), and an accelerometer (e.g., to measure the tilt of the UC apparatus 400). The sensors may be single-use, i.e., configured to be easily replaced by a user after use, or permanent. According to some examples, the UC apparatus 400 may comprise a safety valve (not illustrated) configured to release pressure above a predetermined level, e.g., 5.3 bar.

Reverting to FIG. 4A, the UC apparatus 400 may further comprise one or more user interfaces, configured to receive input and/or display information to a user. The UC apparatus 400 may comprise a data presentation screen 456 configured to display data about the process being undergone, provide instructions to a user, etc. According to some examples, the data presentation screen 456 may be a touchscreen, thereby facilitating its use as a user input device as well as a presentation device. The UC apparatus 400 may further comprise a light unit 458, which may be selectively illuminated to facilitate communication to a user. According to some examples, the light unit 458 may be configured to be selectively illuminated in one of a plurality of colors, for example comprising an RGB or WRGB LED strip, wherein the controller is configured to select the color of illumination thereof to communicate different messages to a user (e.g., a first color may indicate that a process is in progress, a second color may indicate that a process is completed, a third may indicate than an error has occurred, etc.). The UC apparatus 400 may further comprise a speaker configured to facilitate audio communication of one or more messages to a user.

The UC apparatus 400 may be mounted on a control tower, which is generally indicated at 460. The control tower 460 may be configured to rotate the UC apparatus 460, and may further direct operation thereof. In addition, it may be configured to facilitate and/or control input and/or output of fluids thereto.

According to some examples, the control tower 460 comprise a pendulum mechanism 462, configured to grip the UC apparatus 400. The pendulum mechanism 462 may comprise n actuator (not illustrated), for example comprising a stepper motor, configured to selectively pivot the UC apparatus 400 to one or more tilt angles during use.

The control tower 460 may further comprise a plurality of power valves 464, each configured to selectively squeeze and/or release a fluid tube, for example as will be described below.

The control tower 460 may further comprise a plurality of electrical connectors 466. Each of the electrical connectors 466 may be configured to be connected to a component of the UC apparatus 400 and/or the control tower 460, for example to one of the power valves 464, the controller of the UC apparatus 400, the ultrasonic shaker 450, one or more sensors, etc., to supply power and/or facilitate exchange of control signals therewith.

The control tower 460 may further comprise one or more gas connectors 468. Each of the gas connectors 468 may be configured to be connected to one of the ports of the UC apparatus 400 and/or a bioprocess container 470, as will be described below. Moreover, some or all of the gas connectors may be in fluid communication with a source of, and thereby configured to provide, a pressurized gas, including, but not limited to, nitrogen, oxygen, ambient air, vacuum, etc. In order to facilitate supply of gas via the gas connectors 468, the control tower 460 may comprise and/or be configured to direct operation of one or more pumps (not illustrated), e.g., being internal and/or external thereto.

The control tower 460 may further a controller to direct operation of its components.

In use, first and second UC apparatuses 400a, 400b for example each mounted on a respective control tower 460, may be connected in series with one another to carry out a single process, for example wherein the harvest from one of the UC apparatuses is further processed in the other.

For example, as illustrated in FIGS. 4J through 4L, a cell/virus suspension may be supplied from a supply bioprocess container 470a to the upper tank 402 of the first UC apparatus 400a via tube A and the first upper-bottom port 420 (thereby constituting a media-in port). In order to facilitate this, a pressurized gas may be provided to the supply bioprocess container 470a from one of the gas connectors 468. In order to create pressure in the upper tank 402 of the first UC apparatus 400a, a gas, e.g., N₂, may be provided thereto from one of the gas connectors 468 via tube B and the first upper-top port (thereby constituting a gas-inlet port). Venting of the upper tank 402 may be provided via the second upper-top port 418, for example connected to a gas filter 472 attached thereto by tube C. Waste is collected in a first waste bioprocess container 470b via the second upper-bottom port 422 (thereby constituting a concentration-harvest port) of the first UC apparatus 400a) and tube D.

In order to reduce pressure in the lower tank 404 of the first UC apparatus 400a, suction may be provided via the first lower-top port 424 (thereby constituting a gas-suction port) thereof and tube E, which is attached to one of the gas connectors 468. This suction may be utilized to draw the cell/virus suspension into the upper tank 402 via the first upper-bottom port 420 as described above. Venting of the lower tank 404 may be provided via the second lower-top port 426 (thereby constituting a lower venting port), for example connected to a gas filter 472 attached thereto by tube F.

Filtrate which has passed through the filter of the first UC apparatus 400a is collected in the lower tank 404 thereof, and exits via the lower-bottom port 428 (thereby constituting an ultrafiltration-harvest port). It is transferred via tube G, wherein it may be selectively diverted either via tube G′ to a first harvest bioprocess container 470c and/or via tube G″ to the upper tank 402 of the second UC apparatus 400b, via the first upper-bottom port 420 thereof to be further processed therein.

In order to create pressure in the upper tank 402 of the second UC apparatus 400b, a gas, e.g., N₂, may be provided thereto from one of the gas connectors 468 via tube H and first upper-top port 416. Venting of the upper tank 402 may be provided via the second upper-top port 418, for example connected to a gas filter 472 attached thereto by tube J.

Concentrated harvest exits the upper tank 402 of the second UC apparatus 400b via the second upper-bottom port 422 thereof, and may be selectively diverted either via tube K′ to a sample container 474 and/or via tube K″ to a second harvest bioprocess container 470d.

In order to reduce pressure in the lower tank 404 of the second UC apparatus 400b, suction may be provided via the first lower-top port 424 thereof and tube L, which is attached to one of the gas connectors 468. Venting of the lower tank 404 may be provided via the second lower-top port 426, for example connected to a gas filter 472 attached thereto by tube M.

Filtrate which has passed through the filter of the second UC apparatus 400b is collected in the lower tank 404 thereof, and exits via the lower-bottom port 428. It is transferred via tube N to a second waste bioprocess container 470e.

Each of the harvest and waste bioprocess containers 470b, 470c, 470d, 470e may be vented, for example in fluid communication with the environment via a gas filter.

The flow of fluid through each of the tubes A-N may be controlled, e.g., selectively restricted/permitted, by operation of a respective one of the power valves 464.

It will be appreciated that while FIGS. 4J and 4K illustrate all of the connections to the gas connectors 468 being on one of the control towers 470, in practice each of the connections may be made to any suitable gas connector on any one of the control towers, irrespective of which of the UC apparatuses 400 the connection is made, mastitis mutandis.

The UC apparatus 400 may include, according to some embodiments, other features to optimize functionality, such as appropriate inlets, seal assemblies, bearings, plates, ports, gas flow meters for measuring aeration and flow, electronic or manual flow controllers, tachometers to measure RPM of mixer impellers, or other instruments or devices suitable for maintaining, controlling and assessing the environment.

It will be appreciated that the example described above with reference to and as illustrated in FIGS. 4J through 4L is non-limiting, and one or more modifications may be implementing without departing from the scope of the presently disclosed subject matter, mutatis mutandis. For example, as illustrated in FIG. 4M, gas may be removed, e.g., from the lower tank 404 using a vacuum ejector 476 powered by a compressor 478. First, second, and third ejector-control valves 480a, 480b, 480c are provided to selectively connect between, respectively, the vacuum ejector 476 and the compressor 478, the compressor 478 and the lower tank 404, and the lower tank and the vacuum ejector.

In use, the first and third ejector-control valves 480a, 480c may be closed, with the second ejector-control valve 480b remaining open, in order to maintain a positive pressure in the lower tank 404. In order to create a negative pressure in the lower tank 404 and evacuate gas via the vacuum ejector 476, the first and third ejector-control valves 480a, 480c are opened, and the second ejector-control valve 480b is opened.

As illustrated in connection with the second UC apparatus 400b, an addition valve 482 may be provided, for example to selectively seal off the gas pathway when the UC apparatus is tilted for harvesting.

Several control devices may be provided above the upper tank 402, including, but not limited to, some or all of a pressure sensor 484, a liquid level sensor 486, and a safety valve 488.

As an alternative to the selective diverting of concentrated harvest described above with reference to FIGS. 4J through 4L (i.e., in which concentrated harvest exits the upper tank 402 of the second UC apparatus 400b via the second upper-bottom port 422 thereof, and is selectively diverted either via tube K′ to sample container 474 and/or via tube K″ to second harvest bioprocess container 470d), each of the containers may be connected to a different port, e.g., the sample container 474 to the first upper-bottom port 420 and the second harvest bioprocess container 470d to the second upper-bottom port 422, so as to eliminate the “dead zone” between the UC apparatus and the valves which, in the example illustrated in FIGS. 4J through 4L, control access to each of the containers.

In an embodiment, provided herein is a method of filtering macromolecular fluids in the UC apparatus 400, comprising introducing the fluid into the UC apparatus, introducing a gas into the system, and operating the UC apparatus as described in accordance with embodiments herein.

In some embodiments, the UC apparatus 400 is used for separating blood components. In some embodiments, the UC apparatus 400 is used for separating cellular products, for example but not limited to immunoglobulins. In some embodiments, the UC apparatus 400 is used for separating molecules secreted from cells. In some embodiments, the UC apparatus 400 is used for separating viral products produced in cells. In some embodiments, the UC apparatus 400 is used for separating exosomes.

In some embodiments, the supply fluid provided to the system comprises blood. In some embodiments, the supply fluid provided to the system comprises processed blood, e.g., blood that has undergone a process, for example but not limited to apheresis, blood filtration, and blood devoid of coagulating factors. In some embodiments, the supply fluid provided to the system comprises an apheresis product.

### Connection Panel

As illustrated in FIG. 6A, a connection panel, generally indicated at 500, may be provided. The connection panel 500 is configured to facilitate functionally connecting a bioreactor 502 to a control tower 504. It will be appreciated that while the bioreactor 502 illustrated in FIG. 6A is the asymmetric bioreactor described above with reference to and as illustrated in FIGS. 1A through 1G, this is only to illustrate the subject matter described with reference to and as illustrated in FIG. 6A, and should not be construed as limiting the subject matter to any particular device disclosed herein or otherwise, mutatis mutandis. Moreover, it will be appreciated that while the example provided herein refers to a control tower, this is for the sake of illustration only, and in practice the connection panel 500 may be configured for use with any suitable control unit, mutatis mutandis.

Herein the description and appended claims, references to the bioreactor also include auxiliary elements, for example those which control flow of fluids to/from the bioreactor, and/or any other steps involving material processed by or to be processed by the bioreactor. Accordingly, the control panel 500 may be configured to functionally connect the bioreactor 502 with the control tower 504 by connecting elements associated with the bioreactor and which are function as part of its operation.

The connection panel 500 is configured to facilitate connecting one or more electrical elements to the control tower 504. According to some non-limiting examples, the electrical elements comprise one or more sensors configured to detect conditions within the bioreactor 502. The sensors may include one or more of a pO₂ sensor, a pH sensor, a temperature sensor, a glucose sensor, a pressure sensor, a biomass sensor, a lactate sensor, and/or any other suitable sensor. According to other non-limiting examples, the electrical devices comprise one or more power valves 506, each configured to selectively squeeze and/or release a fluid tube, thereby stopping and/or allowing flow therethrough, respectively.

As illustrated in FIG. 6B, the connection panel 500 may comprise a printed circuit board (PCB) comprising a plurality of nodes 508, each configured for electrical connection thereto of one of the electrical elements. Each of the nodes is electrically connected to the control tower 504 for communication therewith. The communication may comprise providing information to the control tower 504, receiving one or more instructions from the control tower, and/or any other suitable communication.

Some of the nodes 508 may be sensor nodes 508a (base reference numeral 508 is used herein without trailing letters to collectively refer to all types of nodes), configured for connection thereto of one end a sensor wire (not illustrated), for facilitating monitoring of the bioreactor 502 and/or its contents. According to some examples, the other end of the sensor wire (i.e., the side not connected to a sensor node 508a) is connected to a probe, a sensor, or is itself a sensor.

Some of the nodes 508 may be power nodes 508b, configured for connection thereto of power and/or control terminals of a device, for example one of the power valves 506, for facilitating control of flow of fluid into and out of the bioreactor 502, for example as described herein.

Accordingly, measurements performed using sensors, probes, etc., attached to the sensor nodes 508a may be transmitted to the control tower 504 via the connection panel 500. Likewise, instructions from the control tower 504 (e.g., operation of a power valve 506) may be transmitted to the relevant element via the connection panel 500.

The PCB may be designed according to any suitable design. According to some examples, the PCB is provided according to a Controller Area Network (CAN bus) architecture, based on International Organization for Standardization (ISO) standard 11898 and/or related standards. According to this standard, all nodes are connected via a single bus comprising two wires, for example a twisted pair. Accordingly, implementation of a CAN bus or similar architecture facilitates providing multiple electrical connections between the control tower 504 and devices connected to the plurality of nodes 508, while reducing the risk of wires becoming entangled, disconnected, etc., as the bioreactor 502 is tilted, pivoted, rotated, etc., for example as described herein.

According to some examples, the connection panel 500 is configured to tilt, pivot, rotate, etc., with the bioreactor 502. Accordingly, wires, cables, tubes, etc., spanning between the connection panel 500 and the bioreactor 502 move with the both the connection panel and the bioreactor, thereby facilitate a reduction and/or prevention of entanglement thereof.

For example, the connection panel 500 may be provided with a through-going slot 510, configured to accommodate therethrough a grip 512 (illustrated in FIG. 6A) of the control tower 504 for gripping the bioreactor 502 and for tilting, pivoting, rotating, etc., it. Accordingly, the grip 512 of the control tower 504 may be accommodated within/through the connection panel 500, allowing the connection panel to move with the grip, thereby facilitating the connection panel tilting, pivoting, rotating, etc., with the bioreactor 502 under control of the grip.

The connection panel 500 may comprise a template on a bioreactor-facing side thereof. The template comprises markings indicating which elements are to be connected to which corresponding node 508.

According to some examples, the template is provided directly on the connection panel 500.

According to some examples, as illustrated in FIG. 6C, the template is provided on a sheet 514, for example made of silicon film or any other suitable material, which may be removably affixed to the connection panel 500. Accordingly, the connection panel 500 may be configured for use with several different types of processes using the bioreactor 502 or using one of several types of bioreactors, wherein a sheet 514 comprising a suitable template for the processes is affixed to the connection panel, and elements connected thereto accordingly.

According to some examples, a kit may be provided, directed toward a particular process. The kit comprises a sheet 514 comprising a template corresponding to the processes (i.e., which indicates how elements are to be connected to the connection panel 500 for performing the processes), and at least some of the elements indicated for connection by the template. The kit may comprise all of the elements, or only disposable elements, e.g., filters, sensors, tubing, the bioreactor, etc. The control tower 504 may be configured to receive information identifying the template, and be programmed to operate accordingly, i.e., based on the connections of the elements to the connection panel 500 as indicated thereby.

### Media Preparation Unit

Reverting to FIG. 6, according to some examples a media preparation unit 530 may be provided. The media preparation unit 530 is configured, inter alia, to heat media to a predetermined temperature before being introduced into the bioreactor 502. Accordingly, media may be stored at a temperature lower than that at which it should be when introduced into the bioreactor 502, e.g., in a cooling unit 520 provided on or near the control tower 504, and only heated just before introduction to the bioreactor. This allows the media to be kept at a storage temperature, and facilitates the control tower 504 to operate to introduce it into the bioreactor 502 only when necessary.

The media preparation unit 530 may be disposed in any suitable location, but in general it may be advantageous for it to be disposed as close to the bioreactor 502 as is practical, in order to reduce the length of tubing through which media must traverse between the media preparation unit and the bioreactor.

As illustrated in FIG. 7, the media preparation unit 530 comprises a main tank 532 for heating therewithin the media, an inlet 534, for example at an upper end thereof, and outlet and 536, for example at a lower end thereof. It further comprises a heating sleeve 538 for receipt therein of a heating element (not illustrated), and a temperature sensor 540. The temperature sensor 540 and/or heating element may be operatively connected to the control tower 504, e.g., via the connection panel for example as described above, for receipt of information from the temperature sensor and/or control of the heating element.

It will be appreciated that while the media preparation unit 530 is illustrated with the connection panel 500, the two are independent of one another, i.e., the connection panel may be provided and/or used without the media preparation unit, and the media preparation unit may be provided and/or used without the connection panel.

In use, media which is to be introduced into the bioreactor 502 is provided to the media preparation unit 530 via the inlet 534, e.g., from the cooling unit 532, to be brought to a suitable temperature for the relevant process before introduction into the bioreactor. The temperature of the media within the main tank 532 is monitored by the temperature sensor 540, while it is heated using the heating element. It will be appreciated that by providing a heating element which extends a significant height of the main tank 532, e.g., more than half of its height, the media therewithin may be more evenly heated compared to a heating element located, e.g., only at the bottom of the main tank, thereby reducing vertical temperature gradients.

When the media within the main tank 532 has reached a suitable predetermined temperature (e.g., as determined by the control tower 504 based on readings taken by the temperature sensor 540), the heated media is provided to the bioreactor 500 via the outlet 536. As mentioned above, the outlet 536 may be located at a bottom end of the main tank 532. This arrangement facilitates removal of media from the media preparation unit 530 for delivery to the bioreactor 502 with a minimal amount of air therewithin.

### Immunoglobulins

In some embodiments, the UC apparatus 400 is used to separate immunoglobulins. A skilled artisan would appreciate that, in some embodiments, blood or processed blood is provided as the supply fluid to the upper tank 402. Further, filter 412 is selected which is permeable to immunoglobulins, which are about 10-15 nm size, but is impermeable to undesired larger molecules. Thus, the immunoglobulins are obtained in the permeate.

In some embodiment, the obtained immunoglobulins comprise antibodies, IgG1, IgG2, IgG3, IgG4, IgM or a combination thereof. In one embodiment, the antibody, a fragment thereof, or combinations thereof have sufficiently high affinity and avidity to their target, which may be a viral protein, a peptide, a nucleic acid, a cancer marker, a cancer protein, a sugar or a combination thereof. In one embodiment, the term “antibody” includes complete antibodies (e.g., bivalent IgG, pentavalent IgM), or fragments of antibodies which contain an antigen binding site. Such fragments include in one embodiment Fab, F(ab′)2, Fv and single chain Fv (scFv) fragments.

In one embodiment, a concentrated hyperimmune globulin appropriate for use in the treatment or prevention of a disease is obtained in the permeate. In another embodiment, the permeate is pooled in appropriately-sized batches and subjected to fractionation procedure which will isolate in one embodiment, and/or purify the immunoglobulin fraction and/or antibodies from the blood components in other embodiments. This is done in one embodiment by the classical Cohn alcohol precipitation method, or a variant thereof, an ion exchange chromatographic method, an affinity chromatographic method, or any other suitable method such as MS-MS (tandem mass spectrometry), LC-MS (preparatory liquid chromatography and mass spectrometry), crystallization or immunopercipitation methods etc. in other embodiments. The final material will be concentrated and the titer or quantity of antibody adjusted as appropriate.

### Exosomes

In some embodiments, the UC apparatus 400 is used to separate exosomes. A skilled artisan would appreciate that, in some embodiments, blood or processed blood is provided as the supply fluid to the upper tank 402. In some embodiments, the filter 412 is permeable to exosomes, and therefore the exosomes are obtained in the permeate. In some embodiments, the filter 412 retains the exosomes in it, and therefore the exosomes are obtained in the filtration medium. In some embodiments, the filter 412 is not permeable to exosomes, and therefore the exosomes are retained in the upper tank 402.

A skilled artisan will appreciate that exosomes are membrane bound extracellular vesicles (EVs) produced in the endosomal compartment of eukaryotic cells. In multicellular organisms, exosomes and other EVs are present in tissues and can also be found in biological fluids including blood, urine, and cerebrospinal fluid. Any of these biological fluids can be provided to the UC apparatus 400. Exosomes are also released in vitro by cultured cells into their growth medium.

The multivesicular body (MVB) is an endosome defined by intraluminal vesicles (ILVs) that bud inward into the endosomal lumen. If the MVB fuses with the cell surface (the plasma membrane), these IL Vs are released as exosomes. Exosomes are usually smaller than most other EVs. According to some examples, they range in diameter between about 50 nm and about 1 μm, for example between about 100 nm and about 400 nm in diameter.

In some embodiments, exosomes carry markers of cells of origin. In some embodiments, exosomes have specialized functions in physiological processes, from coagulation and intercellular signaling to waste management.

In some embodiments, an artificial exosome, which is termed herein also “bioxosome” having all the same qualities, comprises a cell membrane that undergoes fusion with a target cell and releases its cargo into that target cell after the fusion. In some embodiments, the cell membrane component is derived from a selected cellular or extracellular source.

In one embodiment, the exosomes are selective targeting exosomes. In the context of the invention, the term “selective targeting exosomes” refers, without limitation, to exosomes particles with specific targeting ligand or homing moieties. In the selective targeting exosomes of the invention, the ligand or homing moieties are, without limitation, glycosaminoglycan; monospecific or bispecific antibodies; aptamers; receptors; fusion proteins; fusion peptides; or synthetic mimetics thereof; cancer targeting-folic acid; specific phospholipids; cytokines, growth factors; or a combination thereof.

In one embodiment, the membrane of exosomes comprises at least 50% from cell membrane obtained from a cellular source. In one embodiment, the exosomes derived from different sources may show differences in lipid compositions. In some embodiments, exosomes comprise extracellular vesicles.

### Immune Cells

In some embodiments, the UC apparatus 400 is used to separate cells from a liquid medium.

In some embodiments, an immune cell is selected from the group comprising neutrophils, eosinophils (acidophiles), basophils, lymphocytes, and monocytes. In some embodiments, a neutrophil is selected from the group comprising segmented neutrophils and banded neutrophils.

In some embodiments, an immune cell comprises a B cell. In some embodiments, an immune cell comprises a memory B cell. In some embodiments, an immune cell comprises a regulatory B cell (Breg). In some embodiments, an immune cell comprises a T cell. In some embodiments, an immune cell comprises a Killer T cell, or cytotoxic T cell. In some embodiments, an immune cell comprises a Helper T cell. In some embodiments, an immune cell comprises a Th1 cell. In some embodiments, an immune cell comprises a Th2 cell. In some embodiments, an immune cell comprises a Regulatory T cell (Treg).

In some embodiments, an immune cell comprises a monocyte. In some embodiments, an immune cell comprises a dendritic cell. In some embodiments, an immune cell comprises a macrophage. In some embodiments, an immune cell comprises a Myeloid dendritic cell (mDC). In some embodiments, an immune cell comprises a plasmacytoid dendritic cell (pDC). In some embodiments, the compositions disclosed herein comprise more than one type of immune cell.

In some embodiments, an immune cell comprises a memory T cell. In some embodiments, an immune cell comprises a Natural Killer (NK) cell. A skilled artisan would appreciate that NK cells can be identified, for example, by cell surface markers comprising CD16 (FcγRIII), CD57, NKp46.

In some embodiments, the cells separated by the UC apparatus 400 comprises stem cells. In some embodiments, stem cells comprises a hematopoietic stem cells (HSCs), which are the stem cells that give rise to other blood cells by hematopoiesis. A skilled artisan would appreciate that hematopoietic stem cells lack expression of mature blood cell markers and are thus, called Lin−. In addition, Hematopoietic stem cells are characterized by their small size and low staining with vital dyes such as rhodamine 123 (rhodamine lo) or Hoechst 33342 (side population).

In some embodiments, a stem cell comprises colony-forming unit-granulocyte-erythrocyte-monocyte-megakaryocyte (CFU-GEMM). In some embodiments, a stem cell comprises colony-forming unit-lymphocyte (CFU-L). In some embodiments, a stem cell comprises colony-forming unit-erythrocyte (CFU-E). In some embodiments, a stem cell comprises colony-forming unit-granulocyte-macrophage (CFU-GM). In some embodiments, a stem cell comprises colony-forming unit-megakaryocyte (CFU-Meg).

In some embodiments, a stem cell comprises colony-forming unit-basophil (CFU-B). In some embodiments, a stem cell comprises colony-forming unit-eosinophil (CFU-Eos). In some embodiments, a stem cell comprises a mesenchymal stem cell (MSC).

In some embodiments, the blood components separated by the filtration comprise any blood component with therapeutic capabilities.

In some embodiments, a lymphocyte separated by the UC apparatus 400, can be modified, for example in a subsequent process, to express any protein or peptide of interest. In some embodiments, a T-cell is modified to express a T-cell receptor (TCR) having antigenic specificity for a cancer antigen. In some embodiments, a TCRs comprises antigenic specificity for a melanoma antigen. In some embodiments, the antigen comprises gp100 or MART-1. In some embodiments, T-cells are modified to express a chimeric antigen receptor (CAR) having antigenic specificity for a cancer antigen, e.g., any of the cancer antigens described herein.

In some embodiments, lymphocytes are modified to express a cell growth factor that promotes the growth and activation of TIICs, CAR-T cells, or TILs. In some embodiments, a growth factors comprise T-cell growth factors, IL-2, IL-7, IL-15, or IL-12. In some embodiments, modified T-cells express the T-cell growth factor at high levels. T-cell growth factor coding sequences, are readily available in the art, as are promoters, the operable linkage of which to a T-cell growth factor coding sequence promote high-level expression. Suitable methods of modification are known in the art. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Methods for modifying T-cells are known in the art. For example, the T-cells may be transduced to express a TCR having antigenic specificity for a cancer antigen using transduction techniques described in Morgan, et al., Science 314(5796):126-9 (2006) and Johnson et al. Blood 114:535-46 (2009).

In some embodiments, cells separated from the liquid media by the UC apparatus 400 are cultivated in a cell expansion unit by a “pre-Rapid Expansion Protocol (pre-REP),” a “Rapid Expansion Protocol (REP)” step, or a modification thereof. In some embodiments, the pre-REP step, comprises a first expansion of lymphocytes in standard lab media such as RPMI comprising reagents such as irradiated feeder cells and anti-CD3 antibodies. The pre-REP continues until a population of about 5.0×10⁶ cells is reached.

In some embodiments, lymphocytes are plated in a medium, for example RMPI 1640 medium containing 10% human serum, 25 mmol/l HEPES pH 7.2, 100 U/ml penicillin, 100 μg/ml streptomycin, 50 μg/ml gentamycin, 5.5×10⁵ mol/l 2-mercaptoethanol, and 3000 IU/ml IL-2. The medium can be replaced, for example about 1 week after culture initiation.

In some embodiments, lymphocytes are cultured with tumor cells. In some embodiments, lymphocytes are cultured with feeder cells. In some embodiments, for example under a TILs protocol, lymphocytes grow to form a carpet surrounding the tumor cells. In some embodiments, lymphocytes surround the tumor in about 4-7 days, about 7-14 days, or about 14-21 days. In some embodiments, cells are grown and maintained at a concentration lower than about 0.1×10⁶ cells/ml concentration during the pre-REP stage. In some embodiments, cells are grown and maintained at a concentration of about 0.1-0.5×10⁶ cells/ml in the pre-REP stage. In some embodiments, cells are grown and maintained at a concentration of about 0.5-1.0×10⁶ cells/ml in the pre-REP stage. In some embodiments, cells are grown and maintained at a concentration of about 1.0-1.5×10⁶ cells/ml in the pre-REP stage. In some embodiments, cells are grown and maintained at a concentration of about 1.5-2.0×10⁶ cells/ml in the pre-REP stage. In some embodiments, cells are grown and maintained at a concentration of about 2.0-3.0×10⁶ cells/ml in the pre-REP stage. In some embodiments, cells are grown and maintained at a concentration of about 3.0-5.0×10⁶ cells/ml in the pre-REP stage. In some embodiments, cells are grown and maintained at a concentration higher than about 0.1-0.5×10⁶ cells/ml in the pre-REP stage. When cells are confluent, for example at any of the above concentration levels, the culture medium is added.

In some embodiments, the pre-REP stage terminates when a number of less than about 10×10⁶ cells are obtained. In some embodiments, the pre-REP stage terminates when a number of about 10×10⁶ cells are obtained. In some embodiments, the pre-REP stage terminates when a number of about 25×10⁶ cells are obtained. In some embodiments, the pre-REP stage terminates when a number of about 50×10⁶ cells are obtained. In some embodiments, the pre-REP stage terminates when a number of about 75×10⁶ cells are obtained. In some embodiments, the pre-REP stage terminates when a number of about 100×10⁶ cells are obtained. In some embodiments, the pre-REP stage terminates when a number of more than 100×10⁶ cells are obtained.

In some embodiments, any suitable culture medium can be used to grow lymphocytes during REP stage. In some embodiments, lymphocytes are thawed two days before the REP stage and suspended in a lymphocytes culture medium, as TIL-CM. In some embodiments, a TIL-CM comprises some or all of 10% fetal bovine serum, 25 mmol/l HEPES pH 7.2, 100 U/ml penicillin, 100 μg/ml streptomycin, 2 mmol L-glutamine, 5.5×10⁻⁵ mol/l 2-mercaptoethanol in RPMI 1640, and IL-2 3000 IU/ml. In some embodiments, young TILs are then seeded in culture plates and expanded. In some embodiments, cells are cultured in AIM-V cell medium, comprising L-glutamine, 50 μg/ml streptomycin sulfate, and 10 μg/ml gentamicin sulfate.

In some embodiments, expanding the number of lymphocytes may comprise using about 3,000 mL to about 12,000 mL of cell medium. In some embodiments, expanding the number of lymphocytes may comprise using about 4,000 mL to about 11,000 mL of cell medium. In some embodiments, expanding the number of lymphocytes may comprise using about 5,000 mL to about 10,000 mL of cell medium. In some embodiments, expanding the number of lymphocytes may comprise using about 6,000 mL to about 9,000 mL of cell medium.

In some embodiments, cells are seeded in the expansion unit for the REP stage. In some embodiments, lymphocytes are seeded at a concentration lower than about 0.1×10⁶ cells per ml in the REP stage. In some embodiments, lymphocytes are seeded at a concentration between about 0.1-0.5×10⁶ cells per ml in the REP stage. In some embodiments, lymphocytes are seeded at a concentration between about 0.5-1.0×10⁶ cells per ml in the REP stage. In some embodiments, lymphocytes are seeded at a concentration between about 1.0-2.5×10⁶ cells per ml in the REP stage. In some embodiments, lymphocytes are seeded at a concentration between about 2.5-5.0×10⁶ cells per ml in the REP stage. In some embodiments, lymphocytes are seeded at a concentration higher than about 5.0×10⁶ cells per ml in the REP stage.

In some embodiments, lymphocytes are expanded after seeding in the REP stage. In some embodiments, lymphocytes are expanded by about 50-fold during REP stage. In some embodiments, lymphocytes are expanded by about 60-fold during REP stage. In some embodiments, lymphocytes are expanded by about 70-fold during REP stage. In some embodiments, lymphocytes are expanded by about 80-fold during REP stage. In some embodiments, lymphocytes are expanded by about 90-fold during REP stage. In some embodiments, lymphocytes are expanded by about 100-fold during REP stage.

In some embodiments, TILs are expanded by about 100-fold during REP stage. In some embodiments, lymphocytes are expanded by about 100-fold during REP stage. In some embodiments, lymphocytes are expanded by about 200-fold during REP stage. In some embodiments, lymphocytes are expanded by about 300-fold during REP stage. In some embodiments, lymphocytes are expanded by about 400-fold during REP stage. In some embodiments, lymphocytes are expanded by about 500-fold during REP stage. In some embodiments, lymphocytes are expanded by about 600-fold during REP stage. In some embodiments, lymphocytes are expanded by about 700-fold during REP stage. In some embodiments, lymphocytes are expanded by about 800-fold during REP stage. In some embodiments, lymphocytes are expanded by about 900-fold during REP stage.

In some embodiments, lymphocytes are expanded by about 1000-fold during REP stage. In some embodiments, lymphocytes are expanded by about 1500-fold during REP stage. In some embodiments, lymphocytes are expanded by about 2000-fold during REP stage. In some embodiments, lymphocytes are expanded by more than about 2000-fold during REP stage.

In some embodiments, lymphocytes are expanded by stimulating them with an antigen from a cancer cell. In some embodiments, lymphocytes are expanded by stimulating them with human leukocyte antigen A2 (HLA-A2) binding peptide. In some embodiments, lymphocytes are expanded by stimulating them with MART-1:26-35 peptide. In some embodiments, lymphocytes are expanded by stimulating them with gp100:209-217 peptide. In some embodiments, lymphocytes are expanded by stimulating them with NY-ESO-1. In some embodiments, lymphocytes are expanded by stimulating them with TRP-1. In some embodiments, lymphocytes are expanded by stimulating them with TRP-2. In some embodiments, lymphocytes are expanded by stimulating them with tyrosinase cancer antigen. In some embodiments, lymphocytes are expanded by stimulating them with MAGE-A3. In some embodiments, lymphocytes are expanded by stimulating them with SSX-2. In some embodiments, lymphocytes are expanded by stimulating them with VEGFR2. In some embodiments, lymphocytes are expanded by stimulating them with a portion of an antigen. In some embodiment, a lymphocytes stimulating antigen is expressed from a vector.

In some embodiments, lymphocytes are expanded by providing them with a T-cell growth factor. In some embodiments, lymphocytes are expanded by providing them with IL-2. In some embodiments, lymphocytes are expanded by providing them with IL-15. In some embodiments, IL-2 is provided in a concentration of about 3000 IU/mL. In some embodiments, IL-2 is provided in a concentration of about 6000 IU/mL.

In some embodiments, lymphocytes are expanded by providing them with an anti-CD3 antibody. In some embodiments, an anti-CD3 antibody is provided in a concentration of about 30 ng/mL.

In some embodiments, lymphocytes are expanded by providing them with irradiated feeder cells. In some embodiments, feeder cells comprise peripheral blood mononuclear cells (PBMC). In some embodiments, feeder cells comprise irradiated allogeneic feeder cells. In some embodiments, feeder cells comprise irradiated autologous feeder cells. In some embodiments, feeder cells comprise artificial antigen presenting cells. In some embodiments, feeder cells comprise K562 leukemia cells transduced with nucleic acids encoding CD3 and/or CD8.

In some embodiments, irradiated feeder cells are provided at a ratio of about 20:1 feeder cells to lymphocytes. In some embodiments, irradiated feeder cells are provided at a ratio of about 25:1 feeder cells to lymphocytes. In some embodiments, irradiated feeder cells are provided at a ratio of about 50:1 feeder cells to lymphocytes. In some embodiments, irradiated feeder cells are provided at a ratio of about 100:1 feeder cells to lymphocytes. In some embodiments, irradiated feeder cells are provided at a ratio of about 200:1 feeder cells to lymphocytes.

In some embodiments, irradiated feeder cells are provided in a ratio from about 1 to about 20 feeder cells to lymphocytes. In some embodiments, irradiated feeder cells are provided in a ratio from about 20 to about 50 feeder cells to lymphocytes. In some embodiments, irradiated feeder cells are provided in a ratio from about 50 to about 100 feeder cells to lymphocytes. In some embodiments, irradiated feeder cells are provided in a ratio from about 100 to about 200 feeder cells to lymphocytes.

In some embodiments, lymphocytes are grown and maintained at a concentration lower than about 0.1×10⁶ cells/ml in the REP stage. In some embodiments, lymphocytes are grown and maintained at a concentration of about 0.1-0.5×10⁶ cells/ml in the REP stage. In some embodiments, lymphocytes are grown and maintained at a concentration of about 0.5-1.0×10⁶ cells/ml in the REP stage. In some embodiments lymphocytes are grown and maintained at a concentration of about 1.0-2.5×10⁶ cells/ml in the REP stage. In some embodiments, lymphocytes are grown and maintained at a concentration of about 2.5-5.0×10⁶ cells/ml in the REP stage. In some embodiments, lymphocytes are grown and maintained at a concentration higher than about 5.0×10⁶ cells/ml in the REP stage. In some embodiments, lymphocytes are grown and maintained at a concentration of about 1.0×10⁶ cells/ml in the REP stage. When cells are confluent, the contents are split into daughter wells.

In some embodiments, lymphocytes are expanded for about 7 days during the REP stage. In some embodiments, lymphocytes are expanded for about 14 days during the REP stage. In some embodiments, lymphocytes are expanded for about 21 days during the REP stage. In some embodiments, lymphocytes are expanded for less than 7 days during the REP stage. In some embodiments, lymphocytes are expanded for between about 7-14 days during the REP stage. In some embodiments, lymphocytes are expanded for between about 14-21 days during the REP stage. In some embodiments, lymphocytes are expanded for more than 21 days during the REP stage.

### Lymphocytes Efficiency

In some embodiments, the anti-pathogenic activity of lymphocytes is monitored, allowing to select lymphocyte populations particularly efficient for combating a pathogen. In some embodiments, the efficiency of lymphocytes is monitored, allowing to select lymphocyte populations particularly efficient for lysing cancer cells. In some embodiments, lymphocytes capable of lysing cancer cells may be selected by identifying lymphocytes having any suitable trait associated with the lysis of cancer cells. In some embodiments, lymphocytes are selected according to their IFN-γ release upon co-culture with autologous tumor cells. In some embodiments, particularly efficient lymphocytes release about 50 pg/ml or more of IFN-γ upon co-culture with tumor cells. In some embodiments, particularly efficient lymphocytes release about 100 pg/ml or more of IFN-γ upon co-culture with tumor cells. In some embodiments, particularly efficient lymphocytes release about 200 pg/ml or more of IFN-γ upon co-culture with tumor cells. In some embodiments, selected lymphocytes release about 300 pg/ml or more of IFN-γ upon co-culture with tumor cells.

In some embodiments, particularly efficient lymphocytes are further selected or isolated according to cell surface expression. In some embodiments, lymphocytes are selected according to CD8 expression. In some embodiments, lymphocytes are selected according to CD27 expression. In some embodiments, lymphocytes are selected according to CD28 expression. In some embodiments, lymphocytes are selected according to telomere length. Without being bound to a particular theory, it is believed that cell surface expression of one or more of CD8, CD27, and CD28 and longer telomere lengths are associated with positive objective clinical responses in patients and persistence of the cells in vivo.

In some embodiments, the efficiency of lymphocytes is predicted by the aerobic glycolysis of the cells. In some embodiments, the efficiency of lymphocytes is predicted by lactate levels in the cells. In some embodiments, the efficiency of lymphocytes is predicted by pyruvate mitochondrial levels. In some embodiments, the efficiency of lymphocytes is predicted by acetyl-CoA mitochondrial or cytosolic levels. In some embodiments, the efficiency of lymphocytes is predicted by aspartate levels.

In some embodiments, the efficiency of lymphocytes is predicted by nutrient transporter expression and/or activation of the key metabolic regulator mTOR. In some embodiments, the efficiency of lymphocytes is predicted by PI3K activity. In some embodiments, the efficiency of lymphocytes is predicted by Akt phosphorylation. In some embodiments, the efficiency of lymphocytes is predicted by glycolytic enzyme hexokinase II phosphorylation. In some embodiments, the efficiency of lymphocytes is predicted by 4E-BP1 and/or p70S6 kinase phosphorylation. In some embodiments, the efficiency of lymphocytes is predicted by sterol regulatory element-binding protein 2 (SREBP2) activation.

In some embodiments, the efficiency of lymphocytes is predicted by up-regulation of transcription factors c-Myc, estrogen-related receptor α (ERRα), and hypoxia inducible factor-1α (HIF-1α). In some embodiments, the efficiency of lymphocytes is predicted by AP4 expression.

In some embodiments, the efficiency of lymphocytes is predicted by the relative concentrations of oxidized and reduced forms of nicotinamide adenine dinucleotide (NADH and NAD+, respectively). The NAD+/NADH ratio is a component of the redox state of a cell, and reflects the metabolic activities and the health of cells. The effects of the NAD+/NADH ratio on lymphocytes are not fully elucidated. In some embodiments, the NAD+/NADH ratio is measured by sensors interconnected with the UC apparatus 400.

In some embodiments, the efficiency of lymphocytes is predicted by the NAD+/NADH ratio. In some embodiments, a NAD+/NADH ratio below 0.1 is indicative of lymphocytes having high efficiency. In some embodiments, a NAD+/NADH ratio of between about 0.1-0.5 is indicative of lymphocytes having high efficiency. In some embodiments, a NAD+/NADH ratio of between about 0.5-1 is indicative of lymphocytes having high efficiency. In some embodiments, a NAD+/NADH ratio of between about 1-2 is indicative of lymphocytes having high efficiency. In some embodiments, a NAD+/NADH ratio of between about 2-3 is indicative of lymphocytes having high efficiency. In some embodiments, a NAD+/NADH ratio of between about 3-4 is indicative of lymphocytes having high efficiency. In some embodiments, a NAD+/NADH ratio of between about 4-5 is indicative of lymphocytes having high efficiency. In some embodiments, a NAD+/NADH ratio of between about 5-6 is indicative of lymphocytes having high efficiency. In some embodiments, a NAD+/NADH ratio of between about 6-7 is indicative of lymphocytes having high efficiency. In some embodiments, a NAD+/NADH ratio of between about 7-8 is indicative of lymphocytes having high efficiency. In some embodiments, a NAD+/NADH ratio of between about 8-9 is indicative of lymphocytes having high efficiency. In some embodiments, a NAD+/NADH ratio of between about 9-10 is indicative of lymphocytes having high efficiency.

In some embodiments, a NAD+/NADH ratio of between about 10-12.5 is indicative of lymphocytes having high efficiency. In some embodiments, a NAD+/NADH ratio of between about 12.5-15 is indicative of lymphocytes having high efficiency. In some embodiments, a NAD+/NADH ratio of between about 15-17.5 is indicative of lymphocytes having high efficiency. In some embodiments, a NAD+/NADH ratio of between about 17.5-20 is indicative of lymphocytes having high efficiency. In some embodiments, a NAD+/NADH ratio of between about 20-30 is indicative of lymphocytes having high efficiency. In some embodiments, a NAD+/NADH ratio of between about 30-40 is indicative of lymphocytes having high efficiency. In some embodiments, a NAD+/NADH ratio of between about 40-50 is indicative of lymphocytes having high efficiency.

In some embodiments, a NAD+/NADH ratio of about 0.1 is indicative of lymphocytes having high efficiency. In some embodiments, a NAD+/NADH ratio of about 0.5 is indicative of lymphocytes having high efficiency. In some embodiments, a NAD+/NADH ratio of about 2 is indicative of lymphocytes having high efficiency. In some embodiments, a NAD+/NADH ratio of about 3 is indicative of lymphocytes having high efficiency. In some embodiments, a NAD+/NADH ratio of about 4 is indicative of lymphocytes having high efficiency. In some embodiments, a NAD+/NADH ratio of about 5 is indicative of lymphocytes having high efficiency. In some embodiments, a NAD+/NADH ratio of about 6 is indicative of lymphocytes having high efficiency. In some embodiments, a NAD+/NADH ratio of about 7 is indicative of lymphocytes having high efficiency. In some embodiments, a NAD+/NADH ratio of about 8 is indicative of lymphocytes having high efficiency. In some embodiments, a NAD+/NADH ratio of about 9 is indicative of lymphocytes having high efficiency. In some embodiments, a NAD+/NADH ratio of about 10 is indicative of lymphocytes having high efficiency.

In some embodiments, a NAD+/NADH ratio of about 12.5 is indicative of lymphocytes having high efficiency. In some embodiments, a NAD+/NADH ratio of about 15 is indicative of lymphocytes having high efficiency. In some embodiments, a NAD+/NADH ratio of about 17.5 is indicative of lymphocytes having high efficiency. In some embodiments, a NAD+/NADH ratio of about 20 is indicative of lymphocytes having high efficiency. In some embodiments, a NAD+/NADH ratio of about 30 is indicative of lymphocytes having high efficiency. In some embodiments, a NAD+/NADH ratio of about 40 is indicative of lymphocytes having high efficiency.

In some embodiments, the NAD+/NADH ratio is measured by a colorimetric assay. In some embodiments, a NAD+/NADH ratio colorimetric assay is based on an enzymatic cycling reaction in which NAD+ is reduced to NADH, and NADH reacts with a colorimetric probe. In some embodiments, the product of the reaction can be read at 450 nm, wherein the intensity of the product color is proportional to the NAD+ and NADH within a sample. In some embodiments, an acid or base treatment is used to differentiate NADH from NAD+ within a sample. In some embodiments, samples are compared to a known concentration of NAD+ standards. In some embodiments, the optical density (OD) is measured in an ELISA plate reader. In some embodiments, the OD is measured in a closed bioreactor in which lymphocytes are cultivated.

In some embodiments, lymphocytes are harvested when the NAD+/NADH is above a predetermined level. In some embodiments, lymphocytes are harvested when the OD measured at 450 nm is above a predetermined level. In some embodiments, lymphocytes are harvested when the OD measured at 450 nm is below, above, or similar to the OD of a predetermined sample.

Any number of cell cultures growing in the bioreactor or in the cell expansion unit may be examined in parallel for efficiency. In some embodiments, one cell culture is examined for efficiency. In some embodiments, two cell cultures are examined in parallel for efficiency. In some embodiments, three cell cultures are examined in parallel for efficiency. In some embodiments, four cell cultures are examined in parallel for efficiency. In some embodiments, five cell cultures are examined in parallel for efficiency. In some embodiments, more than five cell cultures are examined in parallel for efficiency.

In embodiments in which two or more cultures are examined, the selected cultures may be later combined and the number of lymphocytes further expanded. In some embodiments in which two or more cultures are examined, each examined culture is separately expanded in separate cultures. In some embodiments, expanding multiple examined cultures separately advantageously increases lymphocyte diversity for patient treatment.

A skilled artisan would appreciate that treating a subject with a lymphocyte population particularly efficient for lysing a tumor or combating a pathogen allows reducing the dose of lymphocytes.

### Tumor Infiltrating Immune Cells

In some embodiments, the blood components obtained by the UC apparatus 400 comprise tumor infiltrating immune cells (TIICs). THICs are white blood cells that leave the bloodstream and migrate towards a tumor and try to attack it. In some embodiments, TIICs comprise tumor infiltrating lymphocytes (TILs). In some embodiments, TIICs comprise T cells. In some embodiments, TIICs comprise B cells. In some embodiments, TIICs comprise natural killer (NK) cells. In some embodiments, TIICs comprise macrophages. In some embodiments, TIICs comprise neutrophils. In some embodiments, TIICs comprise dendritic cells. In some embodiments, TIICs comprise mast cells. In some embodiments, TIICs comprise eosinophils. In some embodiments, TIICs comprise basophils. In some embodiments, TIICs comprise plasma cells. In some embodiments, TIICs comprise mature dendritic cells. In some embodiments, TIICs comprise antigen presenting cells (APCs).

In some embodiments, TIICs comprise CD45+ cells. In some embodiments, TIICs comprise CD4+ cells. In some embodiments, TIICs comprise CD8+ cells. In some embodiments, TIICs comprise CD163+ cells. In some embodiments, TIICs comprise CD20+ cells. In some embodiments, TIICs comprise CD3+ cells. In some embodiments, TIICs comprise CD138+ cells. In some embodiments, TIICs comprise CD163+ cells. In some embodiments, TIICs comprise CD56+ cells. In some embodiments, TIICs comprise FoxP3+ cells. In some embodiments, TIICs comprise DC-LAMP+ cells. In some embodiments, TIICs comprise CD28+ cells. In some embodiments, TIICs comprise CD69+ cells.

In some embodiments, TIICs comprise a combination of different types of lymphocytes. A skilled artisan would appreciate that different combinations of lymphocytes have different capacities for killing a tumor. In some embodiments, disclosed herein are specific combinations of lymphocytes particularly potent for killing a tumor.

### Methods of Culturing Immune Cells

According to the methods of the present disclosure, bioreactors described herein may be used for culturing immune cells. The bioreactors described herein are particularly suited for culturing shear sensitive cells.

In some embodiments, disclosed herein is a method for culturing immune cells comprising seeding a population of immune cells in media in the internal cavity of a bioreactor described herein in detail; activating said immune cells, transducing the activated immune cells with a viral vector, expanding the transduced immune cells, and harvesting the transduced immune cells when the desired concentration of cells is obtained.

In some embodiments, immune cells are cultured in a suitable culture media and temperature to permit viability of the cells. As is known to one of skill in the art, different cell types may be grown in different media. The skilled artisan can easily choose which medium is best suited for a particular cell type and/or particular application. In some embodiments, the medium provides all the essential nutrients, including amino acids, carbohydrates, vitamins, minerals, growth factors and hormones, required for optimal culturing of immune cells. In some embodiments, the medium comprises a bicarbonate buffering system in order to permit modulation of pH by CO₂. The medium may contain additional agents that act as a buffer.

In some embodiments, the media comprises one or more cytokines, e.g., such as IL-2, IL-7, IL-9, IL-21, and/or IL-15, or any suitable combination thereof. Illustrative examples of suitable concentrations of each cytokine or the total concentration of cytokines includes about 25 IU/mL, about 50 IU/mL, about 75 IU/mL, about 100 IU/mL, about 125 IU/mL, about 150 IU/mL, about 175 IU/mL, about 200 IU/mL, about 250 IU/mL, about 300 IU/mL, about 350 IU/mL, about 400 IU/mL, about 450 IU/mL, or about 500 IU/mL or any intervening amount of cytokine thereof. In some embodiments, the cell culture medium comprises about 100 IU/mL of each of, or in total of, IL-2, IL-7, IL-9, IL-21, and/or IL-15, or any combination thereof.

Culture conditions vary widely for each cell type, but the artificial environment in which the cells are cultured typically involves the use of a suitable vessel containing: a substrate or, gases (such as O₂, CO₂); and a regulated physio-chemical environment (including regulation of pH, osmotic pressure, temperature, etc.). In general, the temperature should be maintained within a range of about 36 to 39° C. In some embodiments, the temperature is maintained at 37° C.

Culturing immune cells under certain conditions requires oxygenating the media. In some embodiments, the method of culturing immune cells further comprises suppling gas through a gas supply port of the bioreactor. In some embodiments the gas is selected from nitrogen, carbon dioxide, or oxygen. In some embodiments, the gas supplied is nitrogen. In some embodiments, the gas supplied is carbon dioxide. In some embodiments, the gas supplied is oxygen.

In some embodiments, the flow rate of gas into the vessel is 0.1 L/min to 1 L/min. In some embodiments, the flow rate of gas into the internal cavity of the bioreactor described herein is 0.2 L/min. In some embodiments, the flow rate of gas is 0.3 L/min. In some embodiments, the flow rate of gas is 0.4 L/min. In some embodiments, the flow rate of gas is 0.5 L/min. In some embodiments, the flow rate of gas is 0.6 L/min. In some embodiments, the flow rate of gas is 0.7 L/min. In some embodiments, the flow rate 0.8 L/min. In other embodiments, the flow rate is 0.9 L/min.

In some embodiments of the method of culturing immune cells, the level of CO₂ is 8% CO₂ (v/v). In some embodiments, the level of CO₂ is 5% CO₂ (v/v). In some embodiments, the level of CO₂ is 2% CO₂ (v/v). In some embodiments, the partial pressure of dissolved CO₂ or the level of CO₂ in the medium is continuously or intermittently monitored for enabling modulation of the CO₂ level infused, to adjust the concentration to a predetermined value. In some embodiments, the CO₂ level is constant throughout culturing. In some embodiments, the CO₂ level varies throughout culturing.

In some embodiments of the method of culturing immune cells, the dissolved oxygen (DO) is maintained above 10%. In some embodiments, the DO is maintained above 20%. In some embodiments, the DO is maintained above 30%. In some embodiments, the DO is maintained above 40%. In some embodiments, the DO is maintained above 50%. In some embodiments, the DO is maintained above 60%. In some embodiments, the DO is maintained above 70%. In some embodiments, the DO is maintained above 80%. In some embodiments, the DO is maintained above 90%. In some embodiments, the DO level is constant throughout culturing. In some embodiments, the DO level varies throughout culturing. The level of DO is monitored (intermittently or continuously) and can be adjusted according to the particular cell type, culturing stage and/or particular application.

In general, the pH should be maintained within a range of about 6 to 8. In some embodiments, the pH is maintained within a range of about 6.6 to 7.6. In some embodiments, the pH is maintained within a range of about 6.9 to 7.5. In some embodiments, the pH is maintained within a range of about 6.8 to 7.2. In some embodiments, the pH is maintained within a range of about 7.0 to 7.3. In some embodiments, the pH is monitored (intermittently or continuously) and is adjusted to maintain a pH that is optimal for culturing cells. in culture, in some embodiments of the method of the invention, the pH of the culture may be monitored.

A skilled artisan would be familiar with the methods for monitoring culturing conditions, such as the levels of DO, the CO₂ concentration in liquid medium, pH, temperature etc., which are well-known in the art and may be accomplished using commercially available technology.

In some embodiments of the method of culturing immune cells, the bioreactor is tilted at approximately between 5-180°/minute, with a maximum tilt angle being determined by volume. In some embodiments, tilting is at 5°, 6°, 7°, 8°, 9°, 10°, 15°, 20°, 30°, 40°, 50°, 60°, 70°, 80°, 90°, or 100° per minute, or any intervening angle.

In embodiments, the maximum tilt angle can range from about 23° from vertical axis to about 83° from vertical axis. In embodiments, maximum tilt angle is about 23° from vertical axis when the bioreactor is filled with about 15 mL. In embodiments, maximum tilt angle is about 61° from vertical axis when the bioreactor is filled with about 100 mL. In embodiments, maximum tilt angle is about 73° from vertical axis when the bioreactor is filled with about 300 mL. In embodiments, maximum tilt angle is about 83° from vertical axis when the bioreactor is filled with about 500 mL. In some embodiments, the bioreactor is tilted to horizontal position for performing harvest and waste removal steps. In some embodiments, the bioreactor is tilted to vertical position for introduction of transduction agents, transfection mix, etc.

In some embodiments, the method of culturing immune cells comprises seeding a population of immune cells. In some embodiments, the cells are seeded at a density of about 0.1×10⁶ to about 1×10⁷ cells/mL, about 0.1×10⁶ to about 0.9×10⁶ cells/mL, about 0.1×10⁶ to about 0.8×10⁶ cells/mL, about 0.1×10⁶ to about 0.7×10⁶ cells/mL, about 0.1×10⁶ to about 0.6×10⁶ cells/mL, about 0.1×10⁶ to about 0.5×10⁶ cells/mL, about 0.2×10⁶ to about 0.5×10⁶ cells/mL, or about 0.3×10⁶ to about 0.5×10⁶ cells/mL, or any intervening density of cells.

In some embodiments, the method of culturing immune cells comprises activating the immune cells. In some embodiments, the term, “activation” encompasses the state of a T cell that has been sufficiently stimulated to induce proliferation. In some embodiments, activation of T cells is evidenced by the induced cytokine production, and detectable effector functions.

In some embodiments, the method of culturing immune cells comprises activating T cells. In some embodiments, the method of culturing immune cells comprises activating by contacting the T cells with cell culture medium comprising anti-CD3 and anti-CD28 antibodies. In some embodiments, the method of culturing immune cells comprises activating by contacting the T cells with cell culture medium comprising anti-CD3. In some embodiments, the method of culturing immune cells comprises activating by contacting the T cells with cell culture medium comprising anti-CD28 antibodies. In some embodiments, anti-CD3 and/or anti-CD28 antibodies are plate bound anti-CD3 and anti-CD28 antibodies. In some embodiments, anti-CD3 and/or anti-CD28 antibodies are soluble anti-CD3 and anti-CD28 antibodies. In some embodiments, anti-CD3 and/or anti-CD28 antibodies are bead bound anti-CD3 and anti-CD28 antibodies.

In some embodiments, the cell culture medium for activating cells comprises a concentration of anti-CD3 antibody or CD3 binding agent of about 10 ng/mL, about 20 ng/mL, about 30 ng/ml, about 40 ng/ml, about 50 ng/mL, about 60 ng/mL, about 70 ng/mL, about 80 ng/mL, about 90 ng/mL, about 100 ng/mL, or about 200 ng/ml, or any intervening concentration.

In some embodiments, the cell culture medium for activating cells comprises a concentration of anti-CD28 antibody or CD28 binding agent of about 10 ng/mL, about 20 ng/ml, about 30 ng/ml, about 40 ng/ml, about 50 ng/mL, about 60 ng/mL, about 70 ng/mL, about 80 ng/mL, about 90 ng/mL, about 100 ng/mL, or about 200 ng/ml, or any intervening concentration.

In some embodiments, the cell are activated for at least 30 minutes, at least 1 hour, at least 2 hours, at least 3 hours, at least 4 hours, a least 5 hours, at least 6 hours, at least 7 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, or at least 72 hours, or any intervening length of time.

In some embodiments, the method of culturing immune cells comprises transducing the activated immune cells with a viral vector. In some embodiments, the term “transduction” may encompass the delivery of a gene(s) or other polynucleotide sequence, e.g., an engineered TCR or CAR to immune cells, including T cells, using a retroviral or lentiviral vector by means of viral infection. In some embodiments, the method of culturing immune cells comprises transducing the immune cells with a lentiviral vector. In some embodiments, the method of culturing immune cells comprises transducing the immune cells with a retroviral vector. In some embodiments, the viral vector comprises a retroviral vector. In some embodiments, the viral vector comprises a lentiviral vector.

In some embodiments, the viral vector comprises a polynucleotide encoding a chimeric antigen receptor (CAR). In some embodiments, the CAR comprises: an extracellular domain that binds an antigen selected from the group consisting of: alpha folate receptor, 5T4, αvββ integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRVIII, EGP2, EGP40, EPCAM, EphA2, EpCAM, FAP, fetal AchR, FRa, GD2, GD3, *Glypican-3 (GPC3), HL A-A 1+M AGE 1, HLA-A2+MAGE1, HLA-A3+MAGE 1, HLA-A1+NY-ESO-1, HLA-A2+ YESO-1, HLA-A3+NY-ESO-1, IL-1 IRa, IL-13Ra2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2, or any combination thereof; a transmembrane domain derived from a polypeptide selected from the group consisting of: CD8a; CD4, CD28, CD45, PD1, and CD 152; one or more intracellular co-stimulatory signaling domains selected from the group consisting of: CD28, CD54 (ICAM), CD134 (OX40), CD137 (41BB), CD152 (CTLA4), CD273 (PD-L2), CD274 (PD-L1), and CD278 (ICOS); and a CD3C signaling domain.

In some embodiments, the cells are transduced with a vector at an MOI of about 1, 5, 10, 15, 20, 25, 30, 35, 40, 45, 50, 55, 60, 65, 70, 75, 80, 85, 90, 95 or 100, or any intervening integer. In some embodiments, cells are transduced with a vector at a MOI of about 20.

In some embodiments, cells are transduced for about 6 hours, about 12 hours, about 16 hours, about 18 hours, about 24 hours, about 30 hours, about 36 hours, about 42 hours, about 48 hours, about 54 hours, or about 60 hours, or any intervening length of time.

In some embodiments, the one or more transduction agents are added to improve transduction efficiency. In some embodiments, the transduction agent is Hexadimethrine bromide. In some embodiments, Hexadimethrine bromide is added at a concentration of about 6 μg/ml.

In some embodiments, the method of culturing immune cells comprises expanding transduced cells for about 3 days to about 14 days, about 3 days to about 13 days, about 3 days to about 12 days, about 3 days to about 11 days, about 3 days to about 10 days, about 3 days to about 9 days, about 3 days to about 8 days, or about 3 to about 7 days, or about 5 to about 8 days or any intervening number of days.

In some embodiments, the method of culturing immune cells comprises expanding transduced cells for about 3 days, about 4 days, about 5 days, about 6 days, about 7 days, about 8 days, about 9 days, about 10 days, about 11 days, about 12 days, about 13 days, or about 14 days.

In some embodiments, the method of culturing immune cells comprises harvesting said transduced immune cells when the desired concentration of cells is obtained. In some embodiments, harvesting the immune cells comprises one or more of washing the expanded immune cells, centrifuging immune cells, freezing and cryopreserving cells for long term storage. A skilled artisan would be able to determine when the desired concentration of cells is obtained, for example, according to the intended application of the cells, which, in some embodiments, would be the concentration required for adoptive cell therapy.

In some embodiments, the immune cells are expanded at least 5 fold, at least 10 fold, at least 20 fold, at least 30 fold, at least 40 fold, at least 50 fold, at least 100 fold, at least 200 fold, at least 300 fold, at least 400 fold, at least 500 fold, at least 600 fold, at least 700 fold, at least 800 fold, at least 900 fold, or at least 1000 fold, or more compared the starting population of immune cells.

In some embodiments, the method of culturing immune cells further comprises sampling the immune cells from time to time to establish cell counts, viability, cell characterization, for example by FACS analysis, purity, and/or other features of the cells.

In some embodiments, the method of culturing immune cells further comprises adding antigen presenting cells and/or feeder cells.

In some embodiments, the method of culturing immune cells further comprises perfusing fresh culture medium into the bioreactor and removing spent culture medium from the bioreactor. In some embodiments, the method of culturing immune cells further comprises adding fresh media and one or more growth factors. In some embodiments, the one or more growth factors comprises one or more cytokines. In some embodiments, the one or more cytokines are selected from the group consisting of: IL-2, IL-7, IL-15, IL-9, and IL-21.

In some embodiments, the cytokine is IL-2. In some embodiments, the cytokine is IL-7. In some embodiments, the cytokine is IL-15. In some embodiments, the cytokine is IL-9. In some embodiments, the cytokine is IL-21.

In some embodiments, the concentration of IL-2 is between 100 IU/mL to about 300,000 IU/mL. In some embodiments, the concentration of IL-2 is about 100 IU/mL. In some embodiments, the concentration of IL-2 is about 250 IU/mL. In some embodiments, the concentration of IL-2 is about 500 IU/mL. In some embodiments, the concentration of IL-2 is about 1000 IU/mL. In some embodiments, IL-2 is provided in a concentration of about 3000 IU/mL. In some embodiments, IL-2 is provided in a concentration of about 6000 IU/mL. In some embodiments, IL-2 is provided in a concentration of about 300,000 IU/mL.

In some embodiments, the method of culturing immune cells further comprises a step of washing the immune cells. In some embodiments, washing the immune cells comprises tilting said bioreactor toward a horizontal position, increasing pressure inside the bioreactor with nitrogen and extracting media or buffer through the bioreactor waste valve, decreasing pressure inside the bioreactor and tilting the bioreactor toward a vertical position, adding wash buffer, and optionally, repeating washing steps. In some embodiments, a washing step is performed prior to activating the immune cells. In some embodiments, a washing step is performed after activating the immune cells. In some embodiments, a washing step is performed prior to transducing the immune cells. In some embodiments, a washing step is performed after transducing the immune cells. In some embodiments, a washing step is performed prior to expanding the immune cells. In some embodiments, a washing step is performed after expanding the immune cells.

In some embodiments, an immune cell is selected from the group comprising neutrophils, eosinophils (acidophiles), basophils, lymphocytes, and monocytes.

In some embodiments, an immune cell comprises a B cell. In some embodiments, an immune cell comprises a T cell. In some embodiments, an immune cell comprises a Killer T cell. In some embodiments, an immune cell comprises a cytotoxic T cell. In some embodiments, an immune cell comprises a Helper T cell. In some embodiments, an immune cell comprises a T helper 1 (Th1) cell. In some embodiments, an immune cell comprises a Th2 cell. In some embodiments, an immune cell comprises a Regulatory T cell (Treg). In some embodiments, an immune cell comprises an immature T lymphocyte. In some embodiments, an immune cell comprises a mature T lymphocyte. In some embodiments, an immune cell comprises a resting T lymphocyte. In some embodiments, an immune cell comprises an activated T lymphocyte.

In some embodiments, the T cell comprises a CD4+ T cell. In some embodiments, the T cell comprises a CD8+ T cell. In some embodiments, the T cell comprises a CD4+CD8+ T cell. In some embodiments, the T cell comprises a CD4 CD8 cell. In some embodiments, the T cell comprises a TH17 cell. In some embodiments, the T cell comprises a natural killer T cell. In some embodiments, the T cell comprises a naïve T cell. In some embodiments, the T cell comprises a memory T cell. In some embodiments, the T cell comprises a gamma delta T cell. In some embodiments, the T cell comprises a tumor infiltrating lymphocyte (TIL).

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. The specificity of CAR designs may be derived from ligands of receptors (e.g., peptides). In some embodiments, a CAR can target cancers by redirecting the specificity of a T cell expressing the CAR specific for tumor associated antigens.

In some embodiments, the population of immune cells comprises a population of T cells. In some embodiments, the population of T cells comprises a purified population of T cells. In some embodiments, the population of immune cells comprises populations of T cells of a different type. In some embodiments, the population of T cells comprises different subsets of T cells. In some embodiments, the T cells comprise T cell clones that have been maintained in culture for extended periods of time. In some embodiments, the T cells comprise primary T cells.

In some embodiments, the T cells comprise autologous T cells. A skilled artisan would appreciate that the term “autologous” refers to cells obtained from the same individual to which the cells are administered. In some embodiments, the T cells comprise allogeneic T cells. A skilled artisan would appreciate that the term “allogeneic” may encompass cells that are derived from separate individuals of the same species. In some embodiments, allogeneic donor cells are genetically distinct from the recipient. In some embodiments, the APCs comprise syngeneic APCs. A skilled artisan would appreciate that the term “syngeneic” may encompass cells that are genetically similar or identical and hence immunologically compatible. In some embodiments, syngeneic cells are so closely related that transplantation does not provoke an immune response.

In some embodiments, T cells can be from previously stored blood samples, from a healthy individual, or alternatively from an individual affected with a condition. The condition can be an infectious disease, such as a condition resulting from a viral infection, a bacterial infection or an infection by any other microorganism, or a hyperproliferative disease, such as cancer. In some embodiments, the T cells are from a subject suffering from or susceptible to an autoimmune disease or T-cell pathologies. The T cells can be of human origin, murine origin or any other mammalian species.

In some embodiments, the cells obtained by the method of culturing immune cells, comprise CAR T cells. In some embodiments, the cells obtained by the method of culturing immune cells, comprise genetically engineered TCR expressing T cells. In some embodiments, the cells obtained by the method of culturing immune cells, comprise engineered T cells configured for tumor infiltrating lymphocyte (TIL) therapy. In some embodiments, the cells obtained by the method of culturing immune cells, comprise genetically engineered T cells configured for transduced T-cell therapy. In some embodiments, the cells obtained by the method of culturing immune cells, comprise T cells reengineered with a TCR or CAR. In some embodiments, the cells obtained by the method of culturing immune cells, comprise CAR T cells configured for CAR T-cell therapy. In some embodiments, the cells obtained by the method of culturing immune cells, comprise CAR T cells configured for adoptive cell therapy.

In some embodiments, disclosed herein is population of T cells expressing chimeric antigen receptor (CAR) obtained by the method of culturing cells described herein in detail. In some embodiments, the CAR T cells obtained by the method of culturing cells described herein in detail are useful in adoptive cell therapy.

### EXAMPLES

### Example 1: CAR-T Process Utilizing an Asymmetric Bioreactor as Described Herein and Depicted in FIGS. 1A-2

1) Seed cells through the fifth lower-margin port 46e with needleless Luer-lock syringe.
2) Add needed volume of liquid activator (for ImmunoCult CD3/CD28—25 μl of activator per 1 ml of media) and 100 U/ml of IL-2 through the third lower-margin port 46c with a syringe.
3) Turn on soft tilting 5°/minute.
   Maximum tilt angle:
   a. 83° from the vertical axis (when filled with 500 ml);
   b. 73° from the vertical axis (when filled with 300 ml);
   c. 61° from the vertical axis (when filled with 100 ml);
   d. 23° from the vertical axis (when filled with 15 ml).
4) After activation passes through (24-72 hours) tilt the reactor toward harvest/waste horizontal with speed 5°/minute.
5) Open waste valve (second side-cover port 50b) and increase pressure inside of reactor with N₂ through the second housing-side port 48b.

Note: Exact pressure parameters should be defined experimentally, but diapason is 1-2 bar.
6) When all the amount of media is extracted, close the valve and decrease pressure.
7) Fill reactor with pre-heated (37° C.) washing buffer (DPBS) on the half of reactor volume through the second bottom port 44b with 10 ml/min speed.
8) Repeat steps 5-6.
9) Add volume of pre-heated fresh media (AIM-V completed with IL-2) with volume needed to achieve 5.0×10⁶/ml cell density through the second bottom port 44b with 10 ml/min speed.
10) Tilt reactor back to vertical position with speed 5°/minute.
11) Add lentivirus and transduction agent through appropriate ports—the first housing-side 48a and the second lower-margin port 46b—with a syringe. The volume of lentivirus should be counted from virus titer and desired MOI. The volume of Hexadimethrine bromide at 6 μg/ml (transduction agent).
12) Turn on soft tilting 5°/minute.
13) After transduction passes through (16 hours), repeat steps 5-10.
14) Turn on soft tilting 5°/minute and add fresh media (AIM-V completed with IL-2) through the second bottom port 44b with 10 ml/min speed to double it when cell density raises 2.0×10⁶/ml.
15) When desired cell number is achieved (according to cell counting in sample), repeat steps 5-8.
16) Fill reactor with formulation buffer through the fourth lower-margin port 46d (or media through the second bottom port 44b if in current experiment is not GMP QC testing) with volume needed to obtain final product cell concentration (5.0-10×10⁶/ml) with 10 ml/min speed.
17) Open the third side-cover port 50c and increase pressure inside of reactor with N₂ through the second housing-side port 48b.

Note: exact pressure parameters should be defined experimentally, but diapason is 1-2 bar.
18) When harvest is done (look through glass window), close the valve and decrease pressure. The CAR-T process is over.

Recommendation: take samples through the first or fourth housing-side port 48a, 48d using a vacutainer (3-5 ml) safe system twice a day to check the cell viability, number, and transduction efficiency throughout the process.

### Example 2: HEK Process Utilizing an Asymmetric Bioreactor as Described Herein and Depicted in FIGS. 1A-2

1) Fill reactor with 150 ml pre-heated (37° C.) media (MEM containing 10% FBS (heat inactivated), 100 units/ml penicillin/streptomycin and 2 mM l-glutamine) through the second bottom port 44b with 20 ml/min speed.
2) Seed 300×10⁶ cells through the fifth lower-margin port 46e with needleless Luer-lock syringe.
3) Turn on soft tilting 15°/minute (or as determined by the user, for example up to 180°/min). Maximum tilt angle:
   a. 83° from the vertical axis (when filled with 500 ml);
   b. 73° from the vertical axis (when filled with 300 ml);
   c. 61° from the vertical axis (when filled with 100 ml);
   d. 23° from the vertical axis (when filled with 15 ml).
4) Grow cells and add fresh media through the second bottom port 44b with 20 ml/min speed to double it when cell density raises 4.0×10⁶/ml while max reactor volume (450 ml) will be achieved.
5) Optionally:
   a. Tilt the reactor toward harvest/waste horizontal with speed 15°/minute.
   b. Open the second side-cover port 50b and increase pressure inside of reactor with N₂ through the second housing-side port 48b.
   c. When all the amount of media is extracted, close the valve and decrease pressure.
   d. Fill reactor with pre-heated (37° C.) washing buffer (DPBS) on the half of reactor volume through the second bottom port 44b with 20 ml/min speed.
   e. Repeat steps b-c.
   f. Add pre-heated (37° C.) media through the second bottom port 44b with 20 ml/min speed with volume equal to extracted on step c.
6) Tilt reactor back to vertical position with speed 15°/minute.
7) Add transfection mix through the first lower-margin port 46a with a syringe.
8) When transfection is done (6 hrs), repeat steps 5-11.
9) Turn on soft tilting 15°/minute for 48-72 hours to obtain lentivirus.
10) When lentivirus is done (48-72 hours), tilt the reactor toward harvest horizontal with speed 15°/minute.
11) Open the first side-cover port 50a, increase pressure inside of reactor with N₂ through the second housing-side port 48b and harvest media with lentivirus.
12) Then you can close valve and repeat steps 7-8, then harvest cells through the third side-cover port 50c. The HEK process is over.

Recommendation: take samples through the first or fourth housing-side port 48a, 48d using a vacutainer (3-5 ml) safe system twice a day to check the cell viability, number, and transduction efficiency throughout the process.

### Example 3: HEK Protocol for Use with a Bioreactor as Described Herein and Depicted in FIGS. 1A-3E

Steps performed manually are shown in italics.

Note: This protocol is valid for early-stage testing with research-grade reagents. Also, it is suggested it be tested in 500 ml volume reactor and if it is successful move on to a 3 L reactor version.

### HEK Seeding and Cultivation (Day 1)

| | |
|---|---|
| 1.1 | Fill reactor with needed volume of media (150 ml) |
| | (MEM containing 10% FBS (heat inactivated), 100 units/ |
| | ml penicillin/streptomycin and 2 mM l-glutamine.) |
| 1.2 | Seed cells at concentration 0.5 × 10⁶/ml |
| 1.3 | Cultivate cells for 2 days |
| 1.4 | Add 150 ml of media |
| 1.5 | Cultivate cells for 2 days |

### HEK Transfection (Day 5)

| | |
|---|---|
| 2.1 | Optionally: |
| 2.1.1 | Remove media from reactor |
| 2.1.2 | Fill reactor with washing buffer |
| 2.1.3 | Remove buffer from reactor |
| 2.1.4 | Fill reactor with 450 ml of media |
| | (or to achieve 2.0-4.0 × 10⁶ cell density) |

| | |
|---|---|
| 2.2 | Prepare Large-scale transient transfection mix using |
| | 10 ml opti-MEM, plasmid [psPAX2:PMD2J:transfer vector] |
| | ratio of 3:1:3 and PEI MAX at PEI:DNA ratio 3:1, |
| | incubate for 15 min and then add them to the cells in |
| | the reactor |
| 2.3 | Incubate 6 hrs for transfection |
| 2.4 | Repeat steps 2.1.1 through 2.1.4 |
| 2.5 | Incubate in reactor for 48 hrs |

### Lentivirus Harvesting (Day 7)

| | |
|---|---|
| 3.1 | Harvest media from the reactor |
| 3.2 | Harvest virus from the media by centrifugation |
| | at 3500 × g for 5 min at room temperature. |
| 3.3 | Filter the concentrated virus supernatant through |
| | 0.45 um filter, proceed to concentration and |
| | purification step. |
| 3.4 | Concentrate virus |
| 3.5 | Check lentivirus titer by some kit |

### Example 4: CAR-T Protocol for Use with a Bioreactor as Described Herein and Depicted in 1A-3E

Steps performed manually are shown in italics.

Note: This protocol is valid for early stage testing with research-grade reagents

### PBMC: Production (Day 1)

| | |
|---|---|
| 1.1 | Obtain peripheral blood the blood bank in a sterile |
| | infusion bag at ambient temperature. |
| 1.2 | Transfer 25-30 ml blood per 50 ml sterile canonical |
| | tube (two 50 ml tubes). |
| 1.3 | Add to each tube 20 ml DPBS and pipette up and down |
| | several times. |
| 1.4 | Centrifuge the blood samples at 400 × g (acceleration |
| | 9, declaration 5) for 10 min at 18-20° C. |
| 1.5 | Carefully, remove the supernatant using a pipette |
| | without disturbing the white cell layer and |
| | discard it into a waste container. |
| 1.6 | Immediately, resuspend the pellet of each 50 ml |
| | tube with DPBS to a final volume of 50 ml. |
| 1.7 | Split one tube of cells into two tubes (split |
| | 50 ml into 2, 25 ml cells each). |
| 1.8 | Add to each tube 5 ml DPBS and pipette up and |
| | down several times. |
| 1.9 | Invert the Histopaque −1077 bottle several times |
| | to ensure thorough mixing. Add 20 ml Histopaque −1077 |
| | into six 50 ml tubes. |
| 1.10 | Carefully layer the diluted blood (30 ml) onto |
| | the Histopaque −1077 solution. Note: When layering |
| | the blood DO NOT mix the Histopaque −1077 with |
| | the diluted blood). |
| 1.11 | Centrifuge at 800 × g for 30 minutes at room |
| | temperature, with the brake off (acceleration |
| | speed set at 1, deceleration speed set at 0). |
| 1.12 | Remove the upper layer containing plasma and |
| | platelets using a sterile pipette, leaving |
| | the mononuclear cell layer undisturbed at the |
| | interface. |
| 1.13 | Harvest cells by taking the layer of interest |
| | (middle white layer) and transfer into a fresh |
| | new conical tube without disturbing erythrocyte/ |
| | granulocyte pellet. Combine two to three layers |
| | of interest into one fresh tube. |
| 1.14 | Wash the cells twice with DPBS. |

### PBMCs Activation (Day 1)

| | |
|---|---|
| 2.1 | Count the cells. |
| 2.2 | For the generation of CD19-CAR-T transduced T |
| | cells (or PBMC), 2.0 × 10⁸ cells are needed. |
| 2.3 | Pellet the desired number of cells. |
| 2.4 | Resuspend cells in media (AIM V) 200 ml |
| | (at concentration 2.0 × 10⁶ cells/ml) |
| 2.5 | Transfer resuspended cells into reactor. |
| 2.6 | Add ImmunoCult ™ Human CD3/CD28 T Cell |
| | Activator according to manufacturer protoco |
| | and 100 U/ml of IL-2 |

### Lentivirus Transduction (Day 2)

| | |
|---|---|
| 3.1 | Remove media from reactor |
| 3.2 | Add equal volume of washing buffer |
| 3.3 | Remove buffer from reactor |
| 3.4 | Add media till cell density reaches 5.0 × 10⁶/ml |
| 3.5 | Add the Lenti-CD19-CAR-T virus in the desired |
| | multiplicity of infection into the reactor |
| 3.6 | Add Hexadimethrine bromide at 6 ug/ml |
| 3.7 | After 16 hrs., wash cells repeating 3.1-3.4 but |
| | the final cell density up to 2.0 × 10⁶/ml |
| 3.8 | Add 100 u/ml IL-2 at needed volume to start cell |
| | expansion. |

### Cell Expansion (Days 3 to 9)

| | |
|---|---|
| 5.1 | Add media and IL-2 to keep cell density about |
| | 1.0-2.0 × 10⁶/ml until enough cells for |
| | therapy are obtained |
| 5.2 | Prepare the cells for flow cytometry analysis (FACS). |

Cell formulation (Day 10)

| | |
|---|---|
| 6.1 | Wash cells as 3.1-3.3 × 2 |
| 6.2 | Add formulation buffer at needed volume |
| | (depending on cell amount) |
| 6.2 | Take samples for final QC: cell counting, |
| | cell viability by trypan blue staining, |
| | flow cytometry for transduction efficacy. |
| 6.3 | Harvest cell product in formulation bag |

### Example 5: Activated PBMCs Expansion in a Bioreactor Described Herein and Depicted in 1A-3E

PBMCs were thawed and activated on day 0. On day 2 the activated PBMCs were seeded in two bioreactors: the G-Rex10 (Group 1; control) and the Conical bioreactor (BR) (Group 2). Cells were expanded for 7 days.

### PBMCs Thawing and Activation (Day 0)

### PBMCs Seeding (Day 2)

| | |
|---|---|
| 2.1 | Observe cells under inverted microscope and |
| | count cells. |
| 2.2 | Centrifuge cells. |
| 2.3 | Resuspend cells in media: |
| | Group 1-suspend 5 × 10⁶ cells with 40 ml complete |
| | X-VIVO15 medium |
| | Group 2 - suspend 8.65 × 10⁶ cells with 70 ml |
| | complete X-VIVO15 medium (X-VIVO15 medium: |
| | 123.5 ml X-VIVO15 + 6.5 ml EliteGro Adv., + |
| | 130 μl IL-2 (500,000 U/ml) + 162 μl Gentamicin). |
| 2.4 | Transfer resuspended cells into bioreactor and |
| | incubate at 37° C., 5% CO₂. |

### Cell Expansion (Days 3 to 9)

| | |
|---|---|
| 5.1 | Take samples for glucose and lactate measurements, |
| | cell viability and cell count (NucleoCounter |
| | NC-200), on days 4, 7, and 9. |
| 5.2 | Add media and IL-2 on day 4. |

Results: FIGS. 6A and 6B show the total viable cells (FIG. 6A) and lactate concentration (FIG. 6B) for both groups, as measured on days 4, 7 and 9. All cell counts showed a cell viability greater than 95%. The G-Rex10 cells (Group 1) expanded normally (27.6-fold), while the Conical BR cells (Group 2) experienced a lag phase before recovering and expanding (2.5-fold) (FIG. 6A).

It should be appreciated that embodiments formed from combinations of features set forth in separate embodiments are also within the scope of the present invention.

While certain features of the invention have been illustrated and described herein, modifications, substitutions, and equivalents are included within the scope of the invention.

What is claimed is:

1. A bioreactor comprising:
a housing extending along a vertical axis and defining an internal cavity therewithin, and top and bottom covers at opposite ends of the housing; and
a plurality of ports, each configured to facilitate fluid communication between the internal cavity and the exterior of the bioreactor;
wherein the internal cavity has a frustoconical shape, said frustoconical shape being formed such that horizontal cross-sections are coaxial about a line which is angled with respect to the vertical axis.

2. The bioreactor according to claim 1, wherein the frustoconical shape of the internal cavity is parallel to the vertical axis along a single line.

3. The bioreactor according to any one of claims 1 and 2, the housing comprising upper and lower openings, each closed by a respective one of the top and bottom covers, and each being perpendicular to the vertical axis.

4. The bioreactor according to any one of the preceding claims, the housing comprising a vertically extending window.

5. The bioreactor according to any one of the preceding claims, wherein at least some of the ports are configured to facilitate selective fluid communication between the internal cavity and the exterior of the bioreactor.

6. The bioreactor according to any one of the preceding claims, at least one of the ports constituting a liquid injection port facilitating injection of a liquid into the internal cavity.

7. The bioreactor according to claim 6, wherein said liquid injection port comprises a septum membrane providing fluid isolation between the internal cavity and the exterior of the bioreactor, said septum membrane being configured to be facilitate the fluid communication by being pierced by a syringe.

8. The bioreactor according to any one of the preceding claims, at least one of the ports constituting a collection port configured to be connected to an external container.

9. The bioreactor according to claim 8, wherein said collection port comprises a tap connected thereto.

10. The bioreactor according to any one of the preceding claims, at least one of the ports constituting a gas supply port.

11. The bioreactor according to claim 10, wherein the gas supply port comprises a filter.

12. The bioreactor according to any one of the preceding claims, at least one of the ports constituting a needleless access port.

13. The bioreactor according to claim 11, wherein the needleless access port comprises a swabable valve.

14. The bioreactor according to any one of the preceding claims, at least one of the ports constituting a liquid supply port.

15. The bioreactor according to claim 14, wherein said liquid supply port comprises a check valve.

16. The bioreactor according to any one of the preceding claims, further comprising one or more filter membranes.

17. The bioreactor according to claim 16, wherein one or more of said ports is formed in the top or bottom cover, said membrane being disposed between said port and the internal cavity.

18. An apparatus comprising:
an upper tank for receiving therewith a liquid medium and a lower tank in fluid communication with said upper tank via a flow passage;
a plurality of ports, each configured to facilitate fluid communication between the interior and the exterior of the apparatus;
a filter membrane disposed such that liquid flowing from said upper tank to said lower tank mast pass therethrough;
a stirring mechanism configured to mix the contents of the upper tank;
an ultrasonic shaker disposed adjacent the filter; and
a controller configured to direct operation of the stirring mechanism and ultrasonic shaker, and to facilitate passage of liquid from the upper tank to the lower tank.

19. The apparatus according to claim 18, wherein the filter membrane comprises a reinforced filter.

20. The apparatus according to any one of claims 18 and 19, wherein the filter membrane is selected from the group including a microfiltration membrane, an ultrafiltration membrane, and a nanofiltration membrane.

21. The apparatus according to any one of claims 18 through 20, wherein the filter comprises a pore size of 100 kDa NMWCO.

22. The apparatus according to any one of claims 18 through 21, wherein the stirring mechanism comprises a magnetic stirrer.

23. The apparatus according to any one of claims 18 through 22, further comprising a pump configured to facilitate liquid flow through the system.

24. A method of culturing immune cells comprising:
a) seeding a population of immune cells in media in the internal cavity of a bioreactor according to any one of claims 1 through 17;
b) activating said immune cells;
c) transducing said activated immune cells with a viral vector;
d) expanding said transduced immune cells; and
e) harvesting said transduced immune cells when the desired concentration of cells is obtained.

25. The method according to claim 24, further comprising suppling gas through a gas supply port of said bioreactor.

26. The method according to claim 25, wherein said gas is selected from nitrogen, carbon dioxide, or oxygen.

27. The method according to any one of claims 24 through 26, further comprising a step of washing said immune cells.

28. The method according to claim 27, wherein washing said immune cells comprises:
a) tilting said bioreactor toward a horizontal position;
b) increasing pressure inside the bioreactor with nitrogen and extracting media or buffer through bioreactor waste valve;
c) decreasing pressure inside the bioreactor and tilting said bioreactor toward a vertical position
d) adding wash buffer; and
e) optionally, repeating steps (a) to (d).

29. The method according to any one of claims 24 through 28, further comprising adding fresh media and one or more growth factors to the bioreactor.

30. The method according to claim 29, wherein
a) when said bioreactor contains between 300 to 500 ml media, tilting is 83° from the vertical axis;
b) when said bioreactor contains between 100 ml to 300 ml media, tilting is 73° from the vertical axis;
c) when said bioreactor contains between 15 ml to 100 ml media, tilting is 61° from the vertical axis;
d) when said bioreactor contains up to 15 ml media, tilting is 23° from the vertical axis.

31. The method according to any one of claims 24 through 30, wherein the population of immune cells comprises a population of T cells.

32. The method according to any one of claims 24 through 30, wherein the cells obtained comprise CAR T cells configured for adoptive cell therapy.

33. A population of T cells expressing chimeric antigen receptor obtained by the method according to any one of claims 24 through 31.

34. A connection panel configured to facilitate functionally connecting a bioreactor to a control unit, said connection panel being separate from the bioreactor and from the control unit, the connection panel comprising a printed circuit board with a plurality of nodes, each of said nodes being configured for connection to the control unit and connection to an element associated with operation of the bioreactor for functionally connecting it to the control unit.

35. The connection panel according to claim 34, wherein said connection panel comprises a printed circuit board with a plurality of nodes, each of said nodes being configured for connection to an element associated with operation of the bioreactor for communication therewith independently of elements connected to other of said nodes.

36. The connection panel according to claim 35, wherein said nodes are connected to a single bus, said bus being configured to facilitate connection of the connection panel to the control unit.

37. The connection panel according to claim 36, wherein said nodes and bus are provided according to a CAN architecture.

38. The connection panel according to any one of claims 34 through 37, comprising a through-going slot for accommodating therethrough of a grip of the control unit.

39. The connection panel according to any one of claims 34 through 38, comprising a template indicating locations for connections of elements to the connection panel.

40. The connection panel according to claim 39, wherein said template is provided on a sheet configured to be removably affixable to the connection panel.

41. A kit for use with a connection panel according to any one of claims 34 through 38 together with a control unit, the kit being configured to facilitate use of the connection panel and control unit with a bioreactor for performing therein a predefined bioprocess, the kit comprising:
the bioreactor;
a plurality of elements associated with operation of the bioreactor to carry out said bioprocess; and
a sheet being removably affixable to said connection panel, the sheet comprising a template thereon, said template indicating, when suitably affixed to the connection panel, points of connections of said elements to the connection panel, wherein connection of said elements to the connection panel as per the template facilitates operation of said elements by the control tower to carry out said predefined bioprocess.

42. The kit according to claim 41, wherein said elements comprise one or more filters, one or more sensors, and/or one or more tubes.

43. The kit according to any one of the preceding claims, wherein at least some of said elements are disposable.
